(19) United States
(12) Patent Application Publication
(10) Pub. No.: US 2025/0273345 A1
(43) Pub. Date: Aug. 28, 2025

(54) MODEL GENERATION APPARATUS FOR THERAPEUTIC PREDICTION AND ASSOCIATED METHODS AND MODELS

(71) Applicant: GE Healthcare Limited, Chalfont St. Giles (GB)

(72) Inventors: Jan Wolber, Chalfont St Giles (GB); Eszter Katalin Csernai, Budapest (HU); Zoltán Kiss, Budapest (HU); Levente Lippenszky, Budapest (HU); Gergely Horváth, Budapest (HU)

(21) Appl. No.: 18/859,500

(22) PCT Filed: Jun. 3, 2022
(86) PCT No.: PCT/US2022/032084

§ 371 Date: Oct. 23, 2024

Related U.S. Application Data
(60) Provisional application No. 63/335,215, filed on Apr. 26, 2022.

(51) Int. Cl. G16H 50/50; G16H 10/20; G16H 10/40; G16H 20/10; G16H 50/20

(52) U.S. Cl. CPC G16H 50/50; G16H 10/20; G16H 10/40; G16H 20/10; G16H 50/20

(57) ABSTRACT

Methods, apparatus, systems, and articles of manufacture are disclosed for generation and application of models for therapeutic prediction and processing. An example apparatus includes processing circuitry to at least: process input data pulled from a record to form a set of candidate features; train a first model and a second model using the set of candidate features; test the first model and the second model to compare performance of the first model and the second model; select at least one of the first model or the second model based on the comparison; store the selected first model and/or second model; and deploy the selected first model and/or second model to predict a likelihood of at least one of: a) a toxicity occurring due to immunotherapy according to a treatment plan or b) efficacy of the treatment plan for a patient.

### CROSS-REFERENCE TO RELATED APPLICATION

This patent claims the benefit of priority to International Application No. PCT/US2022/032084, filed Jun. 3, 2022, which claims priority to U.S. Provisional Patent Application Ser. No. 63/335,215, filed Apr. 26, 2022, each of which is hereby incorporated herein by reference in its entirety for all purposes.

### FIELD OF THE DISCLOSURE

This disclosure relates generally to model generation and processing and, more particularly, to generation and application of models for therapeutic prediction and processing.

### BACKGROUND

The statements in this section merely provide background information related to the disclosure and may not constitute prior art.

Immunotherapy can be used to provide effective treatment of cancer for some patients. For those patients, immunotherapy can provide higher efficacy and less toxicity than other therapies. Immunotherapy can include targeted antibodies and immune checkpoint inhibitors (ICI), cell-based immunotherapies, immunomodulators, vaccines, and oncolytic viruses to help the patient's immune system target and destroy malignant tumors. However, in some patients, immunotherapy can cause toxicity and/or other adverse side effect. Immunotherapy side effects may be different from those associated with other cancer treatments because the side effects result from an overstimulated or misdirected immune response rather than the direct effect of a chemical or radiological therapy on cancer and healthy tissues. Immunotherapy toxicities can include conditions such as colitis, hepatitis, pneumonitis, and/or other inflammation that can pose a danger to the patient. Immunotherapies also elicit differing (heterogenous) efficacy responses in different patients. As such, evaluation of immunotherapy remains unpredictable with potential for tremendous variation between patients.

### BRIEF DESCRIPTION OF THE DRAWINGS

FIG. 1 illustrates an example model generation apparatus.

FIGS. 2-14 show flow diagrams illustrating execution of instructions to drive operations using the example model generation apparatus of FIG. 1.

FIG. 15 is a block diagram of an example processing platform including processor circuitry structured to execute example machine readable instructions and/or the example operations.

FIG. 16 is a block diagram of an example implementation of the processor circuitry of FIG. 15.

FIG. 17 is a block diagram of another example implementation of the processor circuitry of FIG. 15.

FIG. 18 is a block diagram of an example software distribution platform (e.g., one or more servers) to distribute software (e.g., software corresponding to example machine readable instructions) to client devices associated with end users and/or consumers (e.g., for license, sale, and/or use), retailers (e.g., for sale, re-sale, license, and/or sub-license), and/or original equipment manufacturers (OEMs) (e.g., for inclusion in products to be distributed to, for example, retailers and/or to other end users such as direct buy customers).

In general, the same reference numbers will be used throughout the drawing(s) and accompanying written description to refer to the same or like parts. The figures are not to scale.

As used in this patent, stating that any part (e.g., a layer, film, area, region, or plate) is in any way on (e.g., positioned on, located on, disposed on, or formed on, etc.) another part, indicates that the referenced part is either in contact with the other part, or that the referenced part is above the other part with one or more intermediate part(s) located therebetween.

As used herein, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly that might, for example, otherwise share a same name.

As used herein, “approximately” and “about” modify their subjects/values to recognize the potential presence of variations that occur in real world applications. For example, “approximately” and “about” may modify dimensions that may not be exact due to manufacturing tolerances and/or other real world imperfections as will be understood by persons of ordinary skill in the art. For example, “approximately” and “about” may indicate such dimensions may be within a tolerance range of +/−10% unless otherwise specified in the below description. As used herein “substantially real time” refers to occurrence in a near instantaneous manner recognizing there may be real world delays for computing time, transmission, etc. Thus, unless otherwise specified, “substantially real time” refers to real time+/−1 second.

As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

As used herein, “processor circuitry” is defined to include (i) one or more special purpose electrical circuits structured to perform specific operation(s) and including one or more semiconductor-based logic devices (e.g., electrical hardware implemented by one or more transistors), and/or (ii) one or more general purpose semiconductor-based electrical circuits programmable with instructions to perform specific operations and including one or more semiconductor-based logic devices (e.g., electrical hardware implemented by one or more transistors). Examples of processor circuitry include programmable microprocessors, Field Programmable Gate Arrays (FPGAs) that may instantiate instructions, Central Processor Units (CPUs), Graphics Processor Units (GPUs), Digital Signal Processors (DSPs), XPUs, or microcontrollers and integrated circuits such as Application Specific Integrated Circuits (ASICs). For example, an XPU may be implemented by a heterogeneous computing system including multiple types of processor circuitry (e.g., one or more FPGAs, one or more CPUs, one or more GPUs, one or more DSPs, etc., and/or a combination thereof) and application programming interface(s) (API(s)) that may assign computing task(s) to whichever one(s) of the multiple types of processor circuitry is/are best suited to execute the computing task(s).

In addition, it should be understood that references to “one embodiment” or “an embodiment” of the present disclosure are not intended to be interpreted as excluding the existence of additional embodiments that also incorporate the recited features.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

A large quantity of health-related data can be collected using a variety of mediums and mechanisms with respect to a patient. However, processing and interpreting the data can be difficult to drive actionable results. For example, understanding and correlating various forms and sources of data through standardization/normalization, aggregation, and analysis can be difficult, if not impossible, given the magnitude of data and the variety of disparate systems, formats, etc. As such, certain examples provide apparatus, systems, associated models, and methods to process and correlate health-related data to predict patient outcomes and drive patient diagnosis and treatment. Certain examples provide systems and methods for health data predictive model building. Certain examples provide a framework and machine learning workflow for therapeutic prediction.

For example, immune checkpoints regulate the human immune system. Immune checkpoints are pathways that allow a body to be self-tolerant by preventing the immune system from attacking cells indiscriminately. However, some cancers can protect themselves from attack by stimulating immune checkpoints (e.g., proteins on immune cells). To target cancer cells in the body, Immune Checkpoint Inhibitors (ICIs) can be used to target these immune checkpoint proteins to better identify and attack, rather than hide, cancerous cells.

Despite the great success of ICI cancer treatments, such treatments can pose a great threat to human health, due to their side effect, which is a type of immune-related Adverse Events (irAE) caused by these treatment options. One of these toxicities is hepatitis, which occurs when the liver is affected by the auto-immune-like inflammatory pathological process triggered by ICI. Certain examples predict the onset of irAE hepatitis before the start of the first ICI treatment. More precisely, certain examples predict whether irAE hepatitis will happen in a given time-window after the initiation of the first treatment. Other toxicities such as pneumonitis, colitis, etc., can be similarly predicted.

For example, majority class undersampling is combined with time series data aggregation to obtain a well-balanced and static dataset, which can be fed to the models. Example models include Gradient Boosting (GB) and Random Forest (RF), and/or other models able to accommodate the size and statistical properties of the data. The model can be selected based on an F1 score, which is a measure of the model's accuracy on a dataset. For example, a GB model without undersampling can maximize an F1-score (e.g., harmonic mean of recall and precision), and a RF model with undersampling can provide a high recall (e.g., ratio of true positives found) with relatively low precision (e.g., ratio of true positives among the estimates), which is acceptable due to the cost effectiveness of additional tests required based on the decision of the model. The models are also able to create probability estimates for a label, rather than only the discrete labels themselves.

Input data is prepared to develop and/or feed model(s) to drive prediction, therapy, etc. In certain examples, input is prepared by extracting blood feature information (e.g., a relevant section of blood features, etc.) from Electronic Health Record (EHR) data tables (e.g., received at a processor/processing circuitry from the EHR, etc.), electronic medical record (EMR) information, etc. The blood features are measurements of liver biomarker concentration in blood plasma (such as ALT, AST, Alkaline Phosphatase and Bilirubin, etc.) and other concentration values in the blood. Blood features can be represented as time series data, for example. After blood features are extracted, the time series data is formed into a single complex data structure. The data structure is used to aggregate time series blood feature data into a data table, which can be used with preprocessing and transformation.

For example, feature engineering aggregates the blood feature data by describing the time-series data of the blood particles with an associated mean, standard deviation, minimum, and maximum. Liver features can also be created by taking the last liver biomarker measurements available before treatment. Labels can be created (e.g., using a definition obtained from medical experts, etc.) to classify someone as positive when a level of at least one liver biomarker exceeds a threshold (e.g., 3-times the upper limit of normal, etc.) within a predefined window. Otherwise, a label can classify the patient as negative. A date of an immune checkpoint inhibitor (ICI) treatment can be determined and/or otherwise provided for use with the label and/or the time-series.

After the input data is prepared (e.g., using feature engineering), the dataset is resampled. That is, the dataset resulting from the input preparation is unbalanced. As such, the dataset can be processed to infer, validate, estimate, and/or otherwise resample the prepared feature data in the dataset. For example, random majority class undersampling is performed on the dataset when the goal is to maximize the recall value. When the F1-score is the subject of maximization, then the resampling can be skipped or disregarded.

Using the dataset (e.g., resampled or otherwise), a model can be trained and tested to generate a prediction. For example, when recall maximization is desired, the dataset can be used to train an RF model. When F1-score maximization is desired, the dataset can be used to train a GB model, for example. In certain examples, the trained model can be validated, such as with Leave-One-Out Cross-Validation, where each sample is predicted individually with the rest as the training set.

As such, a variety of “artificial intelligence” (AI) models can be developed and deployed for use in a variety of health prediction applications. For example, a model can be used to predict static and/or dynamic prognostic factors for hepatitis using an AI model and patient (e.g., EHR, etc.) data.

Alternatively or additionally, a predictive model can be developed for ICI-related pneumonitis using small, noisy datasets. Using input data from structured (e.g., EHR, EMR, laboratory data system(s), etc.) and/or unstructured (e.g., curated from laboratory data system(s), EHR, EMR, etc.) data, input features can be evaluated to build models and output a predicted probability of developing pneumonitis. In certain examples, multiple models can be developed, and the system and associated process can iterate and decide between two or more model versions. For example, available data can be divided into two partitions with a sequential forward selection process, and robust performance evaluation can be used to validate and compare two developed models to select one for deployment.

For example, available data can be divided into two partitions, and a sequential forward selection process can be utilized to build a model. In each iteration, two model versions are compared, and the one with higher performance on both partitions is selected. The second model version is created by adding a candidate feature to the first model version. On the larger partition, the comparison is based on performance results in the inner loop of a nested cross-validation (CV). On the smaller partition, permutation test is performed to test whether the candidate feature has predictive power (e.g., the second model version has higher performance). At the end, the outer loop of nested CV is used for robust performance evaluation of the final model.

Certain examples provide an automated framework to prepare EHR and/or other health data for use in machine learning-based model training. For example, the framework prepares data from multiple sources and generates combined time-dependent unrestricted intermediary outputs, which are used to aggregate features for training and deployment of time-dependent models. For example, input data sources can be processed, and the data is used to generate patient vectors. The patient vectors can be used to filter and aggregate, forming an interface definition. As such, a model-agnostic workflow creates input datasets for multiple model training. Intermediary outputs retain temporal structure for sequential modeling tasks and form a maintainable, sustainable framework with interface.

Certain examples provide predictive model building related to ICI, in which input data from multiple sources is prepared. Ground truth prediction labels can be generated from the prepared data and/or labels can be expertly created. Then one or more models are built on a feature matrix generated using labels and data, with ground truth prediction labels as standalone module of the framework. The framework can then drive a workflow to assess multiple efficacy surrogate endpoints to predict response(s) to ICI therapies, for example.

In certain examples, patient health data is prepared and used to train a model using a system involving a plurality of submodules. For example, the system includes a data extraction and processing submodule to extract patient blood test histories from EMR/EHR, clean the blood history data, and perform data quality check(s). A label definition submodule defines one or more feature labels related to the blood history data, and a feature engineering submodule can form blood features by aggregating and processing blood history data with respect to the labels. A model submodule trains and evaluates an AI model to dynamically predict immune-related hepatitis adverse event risk from fixed length blood test histories. Alternatively or additionally, liver function test values can be extracted, cleaned, and organized in a time series. A label definition algorithm can be executed to generate an AI model and target label for each set of blood and/or liver test values, while feature engineering (e.g., normalization, symbolic transformation and motif extraction) can be used train and evaluate AI risk prediction model(s), for example. Similarly, drug history information, medical condition history, anthropometric features, etc., can be used for labeling and feature formation.

Because features can vary significantly between toxicities, toxicity-specific feature formation and model training is important to provide meaningful features and accurate predictive results. Otherwise, a lack of meaningful features destroys performance of the resulting model. Thus, certain examples derive models focused on particular toxicities, and the outputs can be combined (together and/or further with a prediction of efficacy) to form a recommendation, such as based on a risk versus benefit analysis of toxicities versus efficacy for a given immunotherapy drug.

As such, certain examples drive therapy based on a prediction of the likelihood of complications from hepatitis, pneumonitis, etc. Patients can be selected for immunotherapy treatment, be removed from immunotherapy treatment and/or otherwise have their treatment plan adjusted, be selected for an immunotherapy clinical trial, etc., based on a prediction and/or other output of one or more AI models. Model(s) used in the prediction can evolve as data continues to be gathered from one or more patients, and associated prediction(s) can change based on gathered data as well. Model(s) and/or associated prediction(s) can be tailored to an individual and/or deployed for a group/type/etc. of patients, or for a group or individual ICI drug, etc., for example.

In certain examples, data values are normalized to an upper limit of a “normal” range (e.g., for blood test, liver test, etc.) such that values from different sources can be compared on the same scale. Data values and associated normalization/other processing can be specific to a lab, a patient, a patient type (e.g., male/female, etc.), etc. For example, each lab measurement may have a specific normal range that is used to evaluate its values across multiple patients.

With time-series data, one value depends on a previous and/or other value such that the data values have a relationship, rather than being independent. The dependency can be identified and taken into account to identify patients in the data over time. For example, if a data value in a times series of patient blood work exceeds twice a normal limit at a first time, reduces within the normal limit at a second time, and again exceeds twice the normal limit at a third time, then this pattern can be identified as important (e.g., worth further analysis). Data processing can flag or label this pattern accordingly, for example. As such, clinical data from a patient's record can be used over time to identify and form features, anomalies, other patterns, etc. Data is confused to a common model for comparison. Resulting models trained and tested on such data can be robust against outliers, scaling, etc. Features can be created for better (e.g., more efficient, more accurate, more robust, etc.) modeling such as pneumonitis modeling, colitis modeling, hepatitis modeling, etc.

Thus, data processing creates feature(s) that can be used to develop model(s) that can be deployed to predict outcome(s) with respect to patient(s). For example, a data processing pipeline creates tens of thousands of features (e.g., pneumonitis, frequency of ICD-10 codes, frequency of C34 codes, etc.).

For example, data values can include ICD-10 codes for a given patient for a one-year time period. In certain examples, codes can span multiple years (e.g., a decade, etc.) and be harmonized for processing. The ICD-10 codes are processed to identify codes relevant to lung or respiratory function, and such codes can then be used to calculate a relative frequency of lung disease in the patient. As another example, a patient history can be analyzed to determine a relative frequency of a C34 code in the patient history, which is indicative of lung cancer. Smoking status can also be a binary flag set or unset from the data processing pipeline, for example. In certain examples, codes can be converted between code systems (e.g., ICD-9, ICD-10 (such as C34, C78, etc.), etc.). Codes can be reverse-engineered without all of the keys, for example.

Using codes and other data, a plurality (e.g., 5, 6, 10, etc.) of features can be created and used in a modeling framework to predict development of pneumonitis in a patient. The model is built in a stepwise, forward fashion. Labels for pneumonitis models are not inherently in the dataset, so a ground truth is created for model training based on expert judgment to identify labels from patient history(-ies), for example. Codebooks and quality control can be used to correctly label, for example.

In certain examples, historical data received from patients is asynchronous. Systems and methods then align the data for the patient (and/or between patients) to enable aggregation and analysis of the data with respect to a common baseline or benchmark. In certain examples, an influence point or other reference can be selected/determined, and patient data time series/timelines are aligned around that determined or selected point (e.g., an event occurring for the patient(s) such as a check-up, an injury, an episode, a test, a birthdate, a milestone, etc.). For example, a date of first chemotherapy, ICI therapy, first symptom/reaction (e.g., in lung function, etc.), etc., can be used to align patient data.

Processed data can then be used to predict static labels in a predefined or otherwise determined time window, etc. Models can be trained, validated, and deployed for hepatitis, pneumonitis, drug efficacy, etc. As such, data from an EHR, EMR, laboratory system, and/or other data source can be pre-processed and provided to a model to generate a prediction, which can be post-processed and output a user and/or other system for alert, follow-up, treatment protocol, etc. In certain examples, the prediction value is routed to another system (e.g., scheduling, lab, etc.) for further processing.

One or more AI models can be used to facilitate processing, correlation, and prediction based on available patient health data such as blood test results, liver test results, other test results, other patient physiological data, etc. Models can include a high recall low precision model, a low recall high precision model, a harmonic mean maximized (convergence) model, etc. A boosted decision tree model or variant such as random forest (RF), gradient boosting (GB), etc., can be used. For example, a majority class undersampling, random forest model can be used to maximize recall with relatively low precision. However, with hepatitis, prevention is inexpensive and easy, so false negatives can be afforded. As such, a gradient boosting model can be developed to maximize F1 score with no resampling applied.

Machine learning techniques, whether deep learning networks or other experiential/observational learning system, can be used to characterize and otherwise interpret, extrapolate, conclude, and/or complete acquired medical data from a patient, for example. Deep learning is a subset of machine learning that uses a set of algorithms to model high-level abstractions in data using a deep graph with multiple processing layers including linear and non-linear transformations. While many machine learning systems are seeded with initial features and/or network weights to be modified through learning and updating of the machine learning network, a deep learning network trains itself to identify “good” (e.g., useful, etc.) features for analysis. Using a multilayered architecture, machines employing deep learning techniques can process raw data better than machines using conventional machine learning techniques. Examining data for groups of highly correlated values or distinctive themes is facilitated using different layers of evaluation or abstraction.

Throughout the specification and claims, the following terms take the meanings explicitly associated herein, unless the context clearly dictates otherwise. The term “deep learning” is a machine learning technique that utilizes multiple data processing layers to recognize various structures in data sets and classify the data sets with high accuracy. A deep learning network (DLN), also referred to as a deep neural network (DNN), can be a training network (e.g., a training network model or device) that learns patterns based on a plurality of inputs and outputs. A deep learning network/deep neural network can be a deployed network (e.g., a deployed network model or device) that is generated from the training network and provides an output in response to an input.

The term “supervised learning” is a machine learning training method in which the machine is provided already classified data from human sources. The term “unsupervised learning” is a machine learning training method (e.g., random forest, gradient boosting, etc.) in which the machine is not given already classified data but makes the machine useful for abnormality detection. The term “semi-supervised learning” is a machine learning training method in which the machine is provided a small amount of classified data from human sources compared to a larger amount of unclassified data available to the machine.

The term “convolutional neural networks” or “CNNs” are biologically inspired networks of interconnected data used in deep learning for detection, segmentation, and recognition of pertinent objects and regions in datasets. CNNs evaluate raw data in the form of multiple arrays, breaking the data in a series of stages, examining the data for learned features. Hepatitis and/or toxicity can be predicted using a CNN, for example.

The term “recurrent neural network” or “RNN” relates to a network in which connections between nodes form a directed or undirected graph along a temporal sequence. Hepatitis and/or toxicity can be predicted using a RNN, for example.

The term “transfer learning” is a process of a machine storing the information used in properly or improperly solving one problem to solve another problem of the same or similar nature as the first. Transfer learning may also be known as “inductive learning”. Transfer learning can make use of data from previous tasks, for example.

The term “active learning” is a process of machine learning in which the machine selects a set of examples for which to receive training data, rather than passively receiving examples chosen by an external entity. For example, as a machine learns, the machine can be allowed to select examples that the machine determines will be most helpful for learning, rather than relying only an external human expert or external system to identify and provide examples.

The term “computer aided detection” or “computer aided diagnosis” refer to computers that analyze medical data to suggest a possible diagnosis.

Deep learning is a class of machine learning techniques employing representation learning methods that allows a machine to be given raw data and determine the representations needed for data classification. Deep learning ascertains structure in data sets using backpropagation algorithms which are used to alter internal parameters (e.g., node weights) of the deep learning machine. Deep learning machines can utilize a variety of multilayer architectures and algorithms. While machine learning, for example, involves an identification of features to be used in training the network, deep learning processes raw data to identify features of interest without the external identification.

Deep learning in a neural network environment includes numerous interconnected nodes referred to as neurons. Input neurons, activated from an outside source, activate other neurons based on connections to those other neurons which are governed by the machine parameters. A neural network behaves in a certain manner based on its own parameters. Learning refines the machine parameters, and, by extension, the connections between neurons in the network, such that the neural network behaves in a desired manner.

A variety of artificial intelligence networks can be deployed to process input data. For example, deep learning that utilizes a convolutional neural network segments data using convolutional filters to locate and identify learned, observable features in the data. Each filter or layer of the CNN architecture transforms the input data to increase the selectivity and invariance of the data. This abstraction of the data allows the machine to focus on the features in the data it is attempting to classify and ignore irrelevant background information.

Deep learning operates on the understanding that many datasets include high level features which include low level features. While examining an image, for example, rather than looking for an object, it is more efficient to look for edges which form motifs which form parts, which form the object being sought. These hierarchies of features can be found in many different forms of data such as speech and text, etc.

Learned observable features include objects and quantifiable regularities learned by the machine during supervised learning. A machine provided with a large set of well classified data is better equipped to distinguish and extract the features pertinent to successful classification of new data.

A deep learning machine that utilizes transfer learning may properly connect data features to certain classifications affirmed by a human expert. Conversely, the same machine can, when informed of an incorrect classification by a human expert, update the parameters for classification. Settings and/or other configuration information, for example, can be guided by learned use of settings and/or other configuration information, and, as a system is used more (e.g., repeatedly and/or by multiple users), a number of variations and/or other possibilities for settings and/or other configuration information can be reduced for a given situation.

An example deep learning neural network can be trained on a set of expert classified data, for example. This set of data builds the first parameters for the neural network, and this would be the stage of supervised learning. During the stage of supervised learning, the neural network can be tested whether the desired behavior has been achieved.

Once a desired neural network behavior has been achieved (e.g., a machine has been trained to operate according to a specified threshold, etc.), the machine can be deployed for use (e.g., testing the machine with new/updated data, etc.). During operation, neural network classifications can be confirmed or denied (e.g., by an expert user, expert system, reference database, etc.) to continue to improve neural network behavior. The example neural network is then in a state of transfer learning, as parameters for classification that determine neural network behavior are updated based on ongoing interactions. In certain examples, the neural network can provide direct feedback to another process. In certain examples, the neural network outputs data that is buffered (e.g., via the cloud, etc.) and validated before it is provided to another process.

Deep learning machines can utilize transfer learning when interacting with physicians to counteract the small dataset available in the supervised training. These deep learning machines can improve their computer aided diagnosis over time through training and transfer learning. However, a larger dataset results in a more accurate, more robust deployed deep neural network model that can be applied to transform disparate medical data into actionable results (e.g., system configuration/settings, computer-aided diagnosis results, image enhancement, etc.).

One or more such models/machines can be developed and/or deployed with respect to prepared and/or curated data. For example, features can be extracted from EHR/EMR data tables (e.g., liver biomarkers, blood/plasma concentration, etc.), etc. Curated data extracts structured data from unstructured sources (e.g., diagnosis dates from medical notes, etc.), for example. Extracted features form the basis of label creation. Time series measurements are extracted and aggregation produces statistical descriptors for the time series data. A table of such data is then used to train a model to make predictions. In some examples, the data can be resampled if necessary, desired, or determined. The models are validated using a robust cross-validation, such as leave-one-out cross validation.

For example, a selection, input, or target can determine whether to maximize an F1 score or recall with a given model. When F1 score is to be maximized by the model, then no resampling of the data is performed. When recall is to be maximized by the model, then resampling of the data can be performed. The decision can be driven by the deployment environment for the model, for example. When the model is to be deployed in a system facilitating a drug trial, then a F1 score is to be maximized because the drug company wants patients with a highest chance to respond well to a given drug. High recall is more important in a clinical treatment setup where the system wants to eliminate as many toxicities as possible. In a treatment setup for hepatitis, for example, low model precision is acceptable due to the inexpensive nature of treatment for hepatitis.

Associated systems and methods can be used to assess a probability or reliability of a prediction generated by a model. The prediction and associated confidence level or other reliability can be output to another processing system, for example. Probable reactions to immunotherapy treatments can be modeled based on available patient data collected in clinical practice.

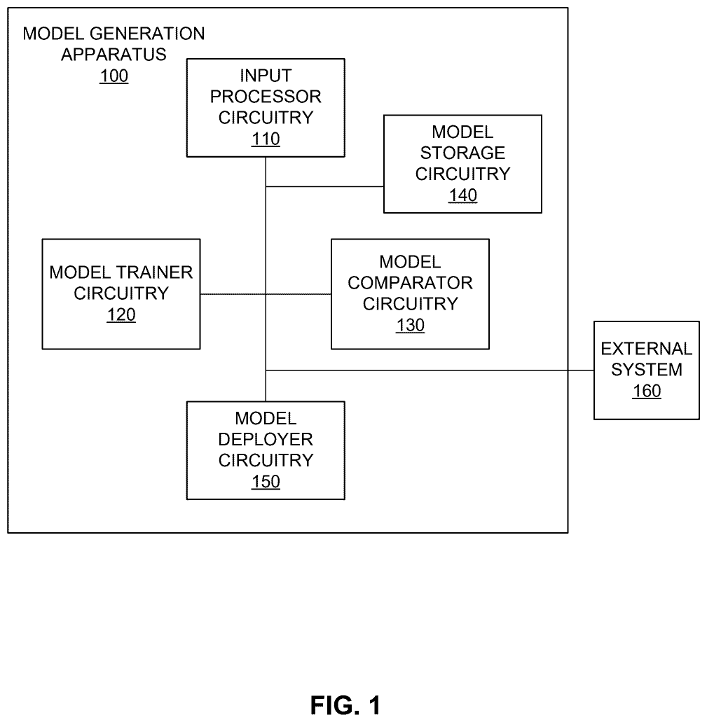

FIG. 1 illustrates an example model generation apparatus 100 including example input processor circuitry 110, example model trainer circuitry 120, example model comparator circuitry 130, example model storage circuitry 140, and example model deployer circuitry 150. As shown in the example of FIG. 1 the example input processor circuitry 110 processes input data pulled from a record to form a set of candidate features. The example model trainer circuitry 120 trains at least a first model and a second model using the set of candidate features. The example model comparator circuitry 130 tests at least the first model and the second model to compare performance of the first model and the second model. Based on the comparison, the example model comparator circuitry 130 selects at least one of the first model or the second model based on the comparison. The example model storage circuitry 140 provides memory circuitry to store the selected first and/or second model. The example model deployer circuitry 150 deploys the selected first model and/or second model to predict a likelihood of a toxicity, such as pneumonitis, hepatitis, colitis, etc., occurring due to immunotherapy according to a treatment plan for a patient.

In operation, the example input processor circuitry 110 processes input data related to one or more patients such as laboratory results, diagnosis codes, billing codes, etc. Input can originate at one or more external systems 160 such as an EHR, EMR, etc. The example input processor circuitry 110 can extract and organize the input in a time series for each patient, for example. In certain examples, the input processor circuitry 110 aligns the input data with respect to an anchor point to organize the input data in the time series. In certain examples, the input processor circuitry 110 generates labels for the input data to form the set of candidate features.

In certain examples, the input processor circuitry 110 performs feature engineering on the set of candidate features and/or underlying data. For example, the input processor circuitry feature engineers the set of candidate features by normalizing, transforming, and/or extracting, for example, from the set of candidate features. In certain examples, the input processor circuitry 110 selects from the set of candidate features to form a patient feature set to train and/or test at least the first model and the second model based on the feature engineering. In certain examples, the input processor circuitry 110 generates a feature matrix to train and/or test/validate the first model and/or the second model based on the feature engineering.

In certain examples, the model deployer circuitry 150 deploys the selected first and/or second model as an executable tool with an interface to facilitate gathering of patient data and interaction with the selected first and/or second model. The deployed model(s) can be used with the tool to select a patient for clinical trial, commence a course of immunotherapy treatment according to a treatment plan or protocol, adjust a current immunotherapy treatment plan, etc. In certain examples, the deployed model(s) can be stored and/or utilized in one more external systems 160 such as EHR, EMR, scheduling system, clinical information system, etc.

As such, the example model generator apparatus 100 can pre-process data from one or more external systems 160 via the example input processor circuitry 110. The example model generator apparatus 100 then trains and validates a plurality of models using the model trainer circuitry 120 and the model comparator circuitry 130. The example model generator apparatus 100 post-processes, stores, and deploys one or more of the trained, validated models using the model storage circuitry 140 and the model deployer circuitry 150. Input data from an EHR, EMR, and/or other external system 160 is transformed into a plurality of models that can be stored and deployed to predict treatment efficacy, risk of toxicity and/or other side effect, etc., from immunotherapy treatment of a particular patient. Data from a plurality of patients can be leveraged through processing and modeling to generate targeted prediction for a particular patient, for example.

In certain examples, the model trainer circuitry 120 trains and validates a boosted decision tree model, other ensemble and/or regression-based learning model, etc. For example, a boosted decision tree model leverages a plurality of weak-learning decision trees to create a strong learning AI model. Trees in the model can correct for errors in other trees of the model, for example, and the entire ensemble of trees in the model works together to generate the predictive output. Models can also be formed as random forest (RF) models, gradient boosted (GB) models, etc. Data is conformed by the model trainer circuitry 120 to a common model for comparison that is robust against outliers and able to be scaled, for example.

In certain examples, the input data processor 110 normalizes input data values to an upper limit of normal such that values (e.g., blood work values, liver function test values, other lab work values, etc.) are on the same scale. Each lab value may have a certain range of values. The input data processor 110 normalizes values for a particular lab type over a range so that the data for that lab is comparable across patients, for example. The input data processor 110 can also organize data into a time series. The time series data can be normalized and/or otherwise adjusted by the input data processor 110 based on an anchor point (e.g., a common or reference event such as hospitalization, symptom, birth, date of diagnosis, date of first ICI administration, etc.).

For example, received patient data may be asynchronous. Selecting an anchor/inflection/influence point allows the data to be aligned for same, similar, and/or different patients. For example, in a patient group with diabetes, ten years of patient history is reviewed while excluding data occurring after diagnosis. The point of diagnosis is selected as an anchor point for a retrospective look at the patient history data (e.g., when no blood sugar levels were being obtained or insulin taken, etc.). As another example, a data of first immunotherapy can be used to align patient data.

As such, the data is organized for identification of relationships and comparisons, for example. Patterns can be identified in the data, for example.

In certain examples, the time series data is formed by the input data processor 110 into a plurality of features. These features can be a set of candidate features from which features are selected to train and validate the models. Feature engineering by the input data processor 110 can form a plurality of features based on codes (e.g., ICD-10 codes, etc.), for example. For example, ICD-10 codes for a patient in a given year can be processed to identify codes relevant to lung and/or respiratory function (C34, C78, etc.), and the time series of those codes can form a function used to calculate a relative likelihood of lung disease in the patient. Similarly, a plurality of features can be formed to predict development of pneumonitis, hepatitis, colitis, and/or other toxicity from immunotherapy treatment.

Features based on lung function, liver biomarkers, blood work (e.g., concentration in blood, plasma, etc.), etc., can form the basis for label and/or other statistical descriptor creation for model training. For example, the gathered data can be compared to a ground truth set of identified labels to generate labels for the data. Resampling can be conducted as well as a validation (e.g., leave-one-out cross validation, other cross-validation, other validation, etc.) to generate a robust model.

The example model trainer circuitry 120 uses the features and/or other data to train one or more AI models, such as a toxicity prediction model (e.g., a hepatitis prediction model, a colitis prediction model, a pneumonitis prediction model, etc.), an efficacy prediction model (e.g., immunotherapy efficacy model, etc.), etc. The models can be high recall with low precision, low recall with high precision, harmonic mean (F1) maximized, majority claim undersampling, etc. A determination can be made by and/or for the example model trainer circuitry 120 (e.g., based on a setting, mode, type, etc.). For example, the model trainer circuitry 120 trains one or more models to maximize the F1 score or to maximize recall. When focusing on F1, resampling may not be necessary. When focusing on recall, resampling (e.g., with a training data set and a test data set, multiple training data sets and/or multiple test data sets, etc.) may be desirable to improve the model.

For example, when configuring participation in a drug trial, a focus is on F1 score maximization to identify patients with a highest chance to respond well to a given immunotherapy drug. When determining a clinical treatment for a patient, however, a goal is to eliminate as many toxicities as possible for the patient. As such, a model developed with high recall is more important, and low precision is acceptable because of inexpensive treatment options for hepatitis, colitis, pneumonitis, etc.

The model comparator circuitry 130 can compare and/or otherwise assess multiple trained models to validate the models and evaluate the probably of the prediction generated by each model. Such assessment/evaluation can be used to select one or more models to be deployed. For example, models can be compared and/or otherwise evaluated based on mean output values, standard deviation of model output, etc. One or more models can be selected for storage in the model storage circuitry 140 and/or other memory circuitry, deployment in a tool via the model deployer circuitry 150, output to one or more external systems 160, etc.

In certain examples, an external system 160 can leverage one or more deployed models from the model deployer circuitry 150 to drive a tool to evaluate efficacy and/or toxicity associated with immunotherapy for a patient and/or patient population, etc. Using one more deployed models, a patient can be assessed at the start of an immunotherapy treatment plan, during administration of the immunotherapy treatment plan, as a candidate for an immunotherapy clinical trial, etc. In certain examples, prior efficacy and/or toxicity model predictions for the patient can be used with updated efficacy and/or toxicity model predictions for the patient (e.g., in combination between prior and current results, with prior as an input to produce current results, etc.).

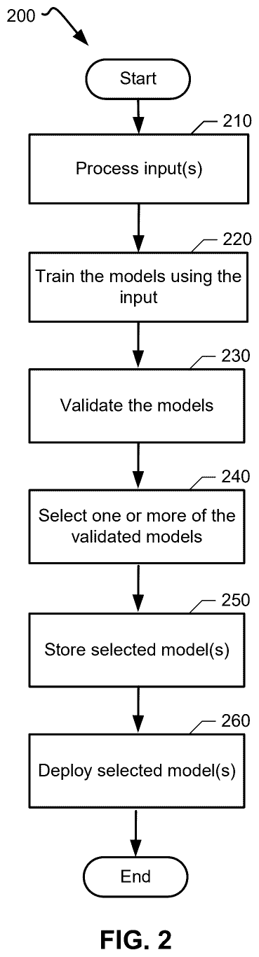

FIGS. 2-14 are flow charts of example processes representing computer-readable instructions storable in memory circuitry and executable by processor circuitry to implement and actuate the example model generation apparatus 100 of FIG. 1. The example process 200 of FIG. 2 begins at block 210, at which the input processor circuitry 110 processes data to form input(s) for the model trainer circuitry 120. For example, the input processor circuitry 110 can align data with respect to an anchor or reference point (e.g., a date, an event, a milestone or other marker, etc.). The input processor circuitry 110 can form a time series from the data. Alternatively or additionally, the input processor circuitry 110 can label and/or perform feature engineering on the data to form an input feature set to train the model(s), for example.

At block 220, models are trained using the input. For example, a plurality of AI models are trained by the model trainer circuitry 120 using the input feature set and/or other input provided from the input processor circuitry 110. By identifying patterns and correlations in the input, the model trainer circuitry 120 forms and weights nodes, layers, connections, etc., in the models (e.g., a RF model, a GB model, a boosted decision tree model, etc.), for example.

At block 230, the trained models are validated by the model trainer circuitry 120. For example, additional input (e.g., features, resampled input data, etc.) is used to validate (e.g., test, verify, etc.) that the trained models work as intended with a threshold level of precision, accuracy, etc.

At block 240, the model comparator circuitry 130 evaluates the validated models to select one or more of the models for deployment. For example, the model comparator circuitry 130 can compare and/or otherwise assess multiple trained, validated (e.g., tested, etc.) models to evaluate the probably of the prediction generated by each model, the suitability of each model for a particular purpose (e.g., prediction of immunotherapy treatment efficacy, prediction of immunotherapy toxicity, immunotherapy clinical trial selection, immunotherapy treatment plan adjustment, etc.). Such assessment/evaluation can be used by the model comparator circuitry 130 to select one or more models. For example, models can be compared and/or otherwise evaluated based on mean output values, standard deviation of model output, etc. In certain examples, the model comparator circuitry 130 selects one model for use/deployment. In other examples, the model comparator circuitry 130 selects a plurality of models (e.g., a toxicity prediction model and an efficacy prediction model, etc.) for use/deployment.

At block 250, the one or more selected models can be stored by the model storage circuitry 140. As such, the selected model(s) can be saved for later use, deployment, etc. In some examples, unselected model(s) can also be stored by the model storage circuitry 140, as the unselected model(s) may be suitable for other usage/deployment in response to a subsequent request.

At block 260, the one or more selected models are deployed by the model deployer circuitry 150 to the external system 160. The model(s) can be deployed as part of a tool for immunotherapy treatment and/or clinical trial planning to provide a toxicity prediction, an efficacy prediction, etc.

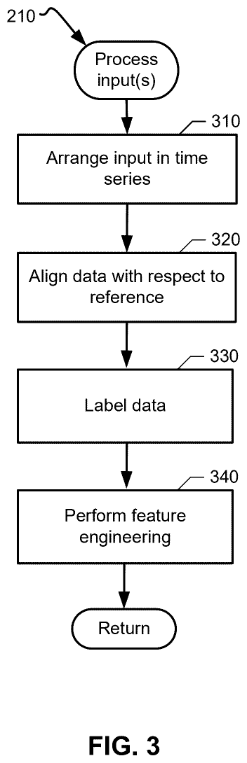

FIG. 3 illustrates an example implementation of input processing for model development (e.g., block 210 of the example of FIG. 2). At block 310, input data for each of a plurality of patients is arranged in one or more time series (e.g., put in order of time to show evolution, progression, dependency, and/or other pattern). At block 320, the time series data for each of the plurality of patients is aligned with respect an anchor point or other reference event. For example, the various sets of time series data can be aligned according to birth, onset of a condition, event such as hospitalization, etc. By aligning the time series, the data in the time series can be more meaningfully evaluated, compared, and/or otherwise analyzed, for example.

At block 330, the aligned time series data is labeled. For example, target labels and associated grades can be generated for each set of data values (e.g., blood test values, liver function values, lung function values, etc.).

At block 340, feature engineering is performed on the data to prepare the data as a set of features with labels to be used to train, test, and/or otherwise validate models. For example, feature engineering can normalize, transform, and extract the data into a set of patient features for application to a model. In certain examples, the features form a set of candidate features that is evaluated to select a certain subset of patient features for model training, validation, etc.

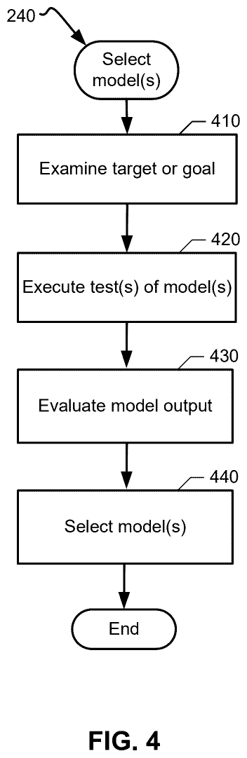

FIG. 4 illustrates an example implementation of model selection for deployment (e.g., block 240 of the example of FIG. 2). At block 410, a target or goal is examined. For example, a request for AI model may be for clinical trial evaluation, immunotherapy efficacy prediction, immunotherapy-associated toxicity and/or other side effect prediction, etc. Such a request can drive selection of a model with a certain characteristic such as high F1 score, high recall, etc. Based on the requested target or goal, a certain subset of available models can be selected. Put another way, based on the requested target or goal, a certain subset of available models can be excluded, leaving a subset of models available for further evaluation and selection.

At block 420, a test is executed using each remaining model. For example, a permutation test, binomial test, and/or other comparative operation is executed using each model (e.g., evaluating lung function models with respect to smoking, no smoking, other binomial and/or permutation test, etc.). At block 430, an output of each model executing the test is evaluated based on one or more criterion. For example, each model can be evaluated based on mean, standard, and/or other comparative criterion to determine which model best satisfies the one or more criterion.

At block 440, based on the comparison of output and/or other model performance, one or more models are selected. For example, a single model may be selected for deployment based on the evaluation of how the model performed and/or output with respect to the one or more criterion. In some examples, multiple models may be selected based on the one or more criterion and/or other factor. For example, a plurality of models may satisfy the criterion(-ia). As another example, a request may include a request for treatment efficacy as well as likelihood of associated toxicity. In response to such a request, both an efficacy model and a toxicity model can be selected. In some examples, a single model may be trained on both toxicity and efficacy to output a combined prediction.

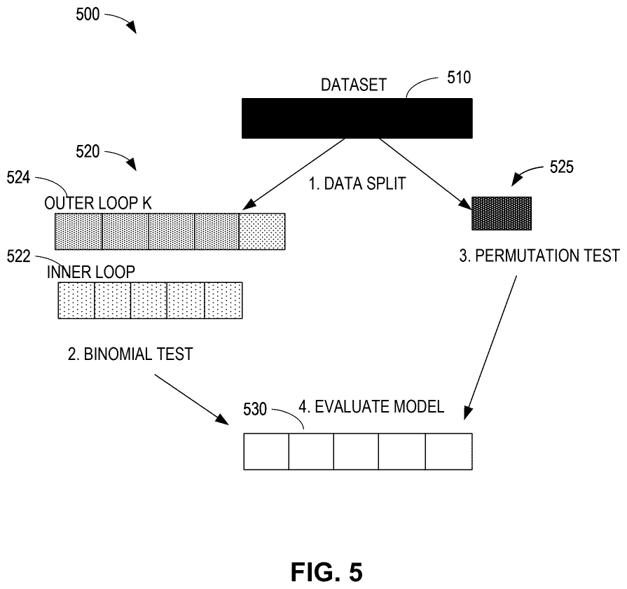

FIG. 5 illustrates an example sequential procedure 500 for model building and evaluation (e.g., through development of a feature set, etc.). As shown in the example of FIG. 5, at 1, an initial data set 510 is split into at least two partitions. Separate data analysis is performed on a first partition of the data 520 and a second partition of the data 525. The first data partition 520 is divided into at least two loops, such as an inner loop 522 and an outer loop 524 for data analysis. At 2, a first test, such as a binomial test evaluates data values in the inner loop 522 and the outer loop 524 (e.g., reject if a data value H₀ includes “smoking”, etc.) to train a model. At 3, the separate, “no touch” test data set 525 is processed using a permutation test, rather than using separate loops. The data set 525 can be processed over multiple permutations with a criterion such as with smoking versus without smoking to identify a likelihood of smoking greater than without smoking, for example. At 4, the model is evaluated to report mean, standard, and/or other evaluation/performance statistic with respect to the first and second data partitions 520, 525 and associated tests.

In certain examples, one or more of the loops 522, 524 are used to evaluate the first data partition 520 via exploratory data analysis with a K-fold cross-validation (CV) comparing a first feature set M1 and a second feature set M2. Through sequential iteration, a feature F is added to the first feature set M1 if K-fold CV performance of M1 is less than M2. Once features have been evaluated accordingly, the resulting model performance is tested using the second data partition 525. If both loops 522, 524 are utilized, M1 and M2 can be compared based on an inner loop 522 result (e.g., a binomial test). When M2 is chosen, a permutation test is performed to assess whether M2 is sufficiently better than M1 on the validation data set 525. Then, model performance can be evaluated on the outer loop 524, for example. As such, an initial feature set, M1, or an extended feature set, M2, can be selected for model training.

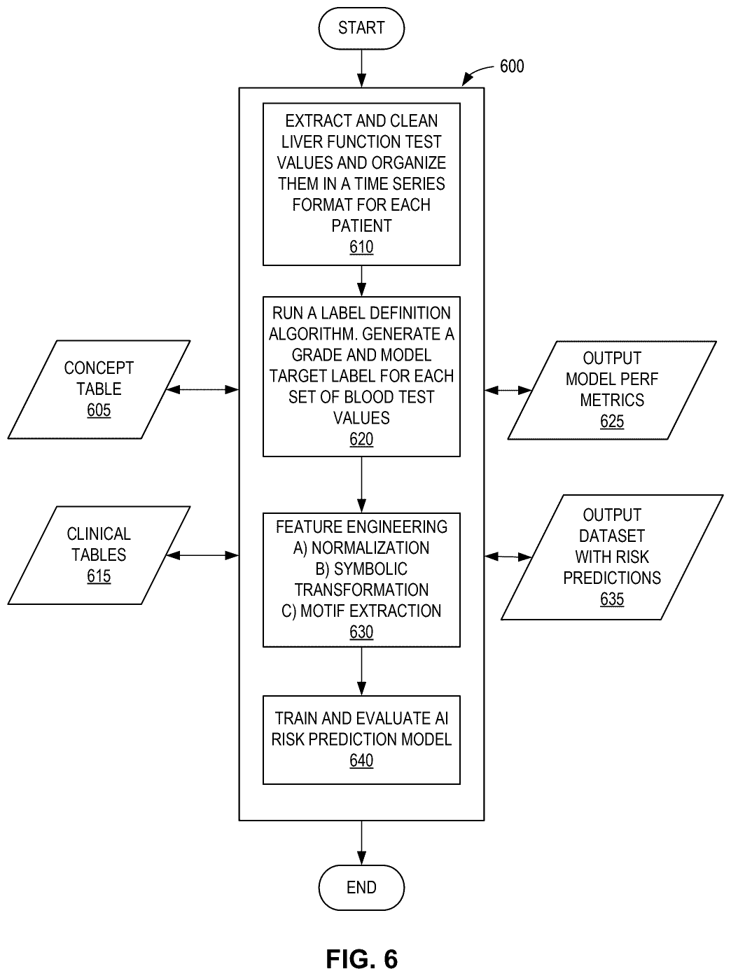

FIG. 6 illustrates an example process 600 for preparing input data for model training. Using the example process 600, data from disparate observational databases can be transformed into a common format (e.g., data model) and common representation (e.g., common terminology, vocabulary, coding scheme, etc.) for systematic analysis according to a library of analytic routines. The example process 600 accepts an input of concept table(s) 605 including concept identifiers, concept names, concept descriptions, etc. For example, concepts in this example relate to liver function tests. Clinical tables 615 are also input with database extracts for patient demographics, hospital visits, laboratory measurements, etc. At block 610, patient liver function test values (e.g., represented in patient blood test histories) are extracted from electronic medical records, etc., cleaned, quality checked, and organized in a time series format for one or more patients. Such extraction and processing can also apply to other patient information (e.g., lung function, drug efficacy, etc.).

At block 620, a label definition algorithm is executed to assign an adverse event (AE) grade to the set of blood test/liver function values (e.g., ALT, AST, TBILIRUBIN, ALKPHOS, etc.) and create a binary target label for the AI model. Such label definition can also apply to other patient test values (e.g., lung function, etc.).

At block 630, feature engineering is performed. For example, blood test values and/or other values can be normalized to an upper limit of “normal” to aid in feature generation, comparison, and other analysis. Such a “normal” range can be determined for a particular patient, based on particular blood work, other laboratory test, etc. Feature engineering transforms the normalized values into a discretized symbolic representation, such as a modified Symbolic Aggregate ApproXimation, etc. In certain examples, motifs can be extracted as n-grams from the series of symbol representations, and counts from patient history can be used as features.

At block 640, an AI model can then be trained and evaluated based on the features, etc., to dynamically predict immune-related adverse event risk from patient data. For example, immune-related adverse event risk can be predicted by the model from fixed-length blood test histories, etc. Output can include model performance metrics 625, a data set of AE risk prediction 635, etc. For example, the risk prediction dataset 635 can include immune-related hepatitis adverse event risk prediction for each history of blood test values. That is, for each patient, a risk of developing a hepatitis AE is predicted until a next ICI treatment appointment for that patient based on their blood test history, etc. The dataset 635 can alternatively include risk prediction for pneumonitis, colitis, hospitalization, etc., based on associated patient data training the model.

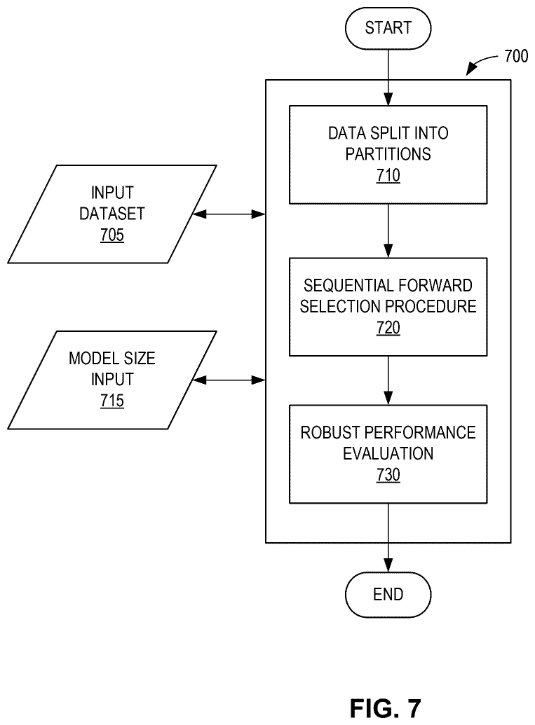

FIG. 7 illustrates an example process 700 for building an example classification model. At block 710, data from an input dataset 705 is divided into a plurality of partitions. For example, structured EHR-derived tabular data, unstructured data tables, aggregated measurement features over time (e.g., 30 days, 60 days, 1 year, etc.), curated labels (e.g., pneumonitis labels, hepatitis labels, colitis labels, etc.), other features (e.g., condition, smoking status, etc.) aggregated over time (e.g., 30 days, 60 days, 1 year, etc.), etc., can be gathered and partitioned. For example, 90% of available data can be organized in a first partition, and the remaining 10% of the data can be organized in a second partition. A model size input 715 can help adjust partitions, etc., based on a typical, expected, or desired number of features in a model.

At block 720, a sequential forward selection procedure is executed to evaluate a plurality of models. The example procedure iterates to find patterns in the data to form potential predictive features. For example, the first partition can be evaluated to identify candidate features based on associations between labels and features. A model is selected on each iteration, and the evaluation continues.

At block 730, model performance is evaluated. For example, a model selected on a final iteration of the selection procedure 720 is cross-validated, such as on an outer loop of a nested cross-validation using the input dataset 705.

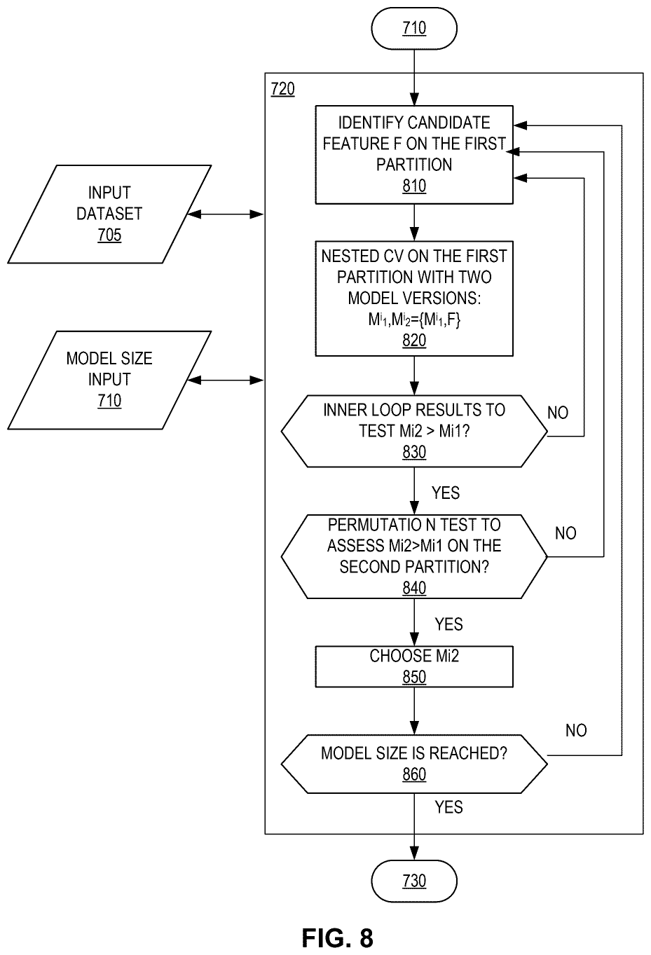

FIG. 8 provides an example implementation of the sequential forward selection procedure 720 of the example process 700 of FIG. 7. At block 810, a candidate feature F is identified in the first data partition. For example, the candidate feature F can be identified based on associations between a target label and existing/identified features. At block 820, a nested cross-validation (CV) is performed on the first partition with two models, M₁ⁱ, M₂ⁱ={M₁ⁱ, F}. That is, model M₂ⁱ includes the features of model M₁ⁱ plus new candidate feature F.

At block 830, inner loop results are evaluated to test whether M₂ⁱ is better than M₁ⁱ. For example, a binomial test is performed to assess whether M₂ⁱ is better than M₁ⁱ based on the inner loop results. If not, then control returns to block 810 to identify another candidate feature in the first partition. When M₂ⁱ is better than M₁ⁱ, at block 840, a permutation test is executed to assess whether M₂ⁱ is better than M₁ⁱ on the second partition. For example, M₂ⁱ and M₁ⁱ can be evaluated based on a p-value, which represents a fraction of permutations of the candidate feature F in which the performance of M₁ⁱ is not smaller than the performance of M₂ⁱ. When M₂ⁱ performs “significantly better” than M₁ⁱ. (e.g., with a standard significance level defined as 0.05 or 0.1), then, at block 850, model M₂ⁱ is selected. Otherwise, control returns to block 810 to identify another candidate feature in the first partition.

At block 860, model size is evaluated (e.g., by comparing current feature set to the model size input 715, etc.). If model size has been reached, then the process can advance to block 730. However, if the model size is not yet met, control returns to block 810 to select a new candidate feature in a next iteration of the example process.

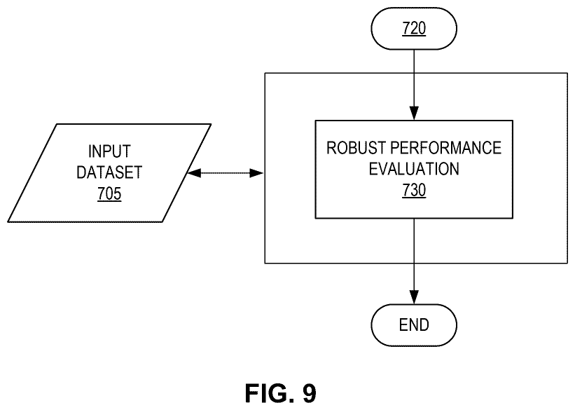

FIG. 9 provides an example implementation of the robust performance evaluation 730 of the example process 700 of FIG. 7. At block 730, performance of the final selected model M₁ⁱ or M₂ⁱ is evaluated using nested cross-validation (CV). As shown in the example of FIG. 9, the input dataset 705 serves as an external test set to validate performance of the selected model and its features. As such, an inner loop of partitioned data can be used to evaluate various candidate models, and an outer loop of test data can be used to validate the performance of the selected model, for example.

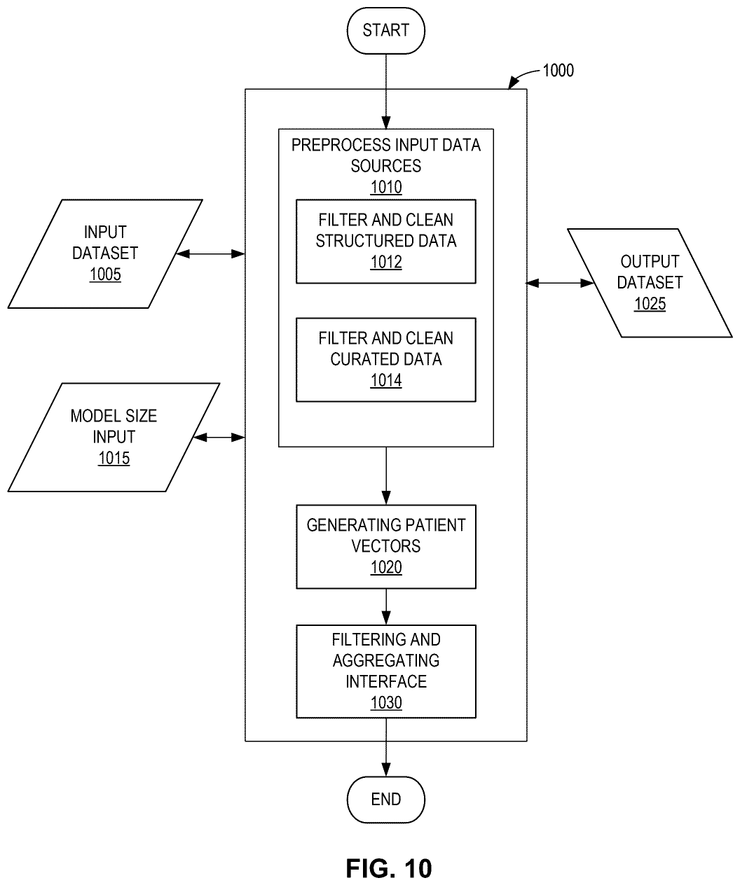

FIG. 10 illustrates an example process 1000 to prepare data from a plurality of sources for use in machine learning and/or other AI model training. For example, database extraction and/or expert curation can be used to generate data from/in an input dataset 1005. Data can be gathered from multiple structured and/or unstructured data sources to form the dataset 1005.

At block 1010, one or more input data sources 1005 are preprocessed to prepare the input data (e.g., by cleaning and extracting features from the data, labeling the features, etc.). Input data and resulting features can include smoking history, drug administration, medical condition(s), radiotherapy history, laboratory measurement(s), anthropometric data, etc. For example, at block 1012, structured data (e.g., drug administration, medical conditions, laboratory measurements, clinical procedures, anthropometric data, etc.) from the input dataset 1005 is filtered and cleaned in a common data format (e.g., a common data model such as an OMOP data model format, etc.). At block 1014, curated data (e.g., related to downstream tasks, smoking history, specific condition history, specific medical history, etc.) from the input dataset 1005 is filtered and cleaned to the common data format.

The preprocessed data set forms an input to block 1020, at which patient vectors are generated from the filtered, cleaned aggregated data set. For example, a model-agnostic set of aggregated patient vectors can be provided with feature filtering. Model-agnostic unaggregated tables having temporal data structure can be used for feature filtering and vector generation. The patient vectors and/or the unaggregated tables can form part of an output dataset 1025.

At block 1030, an interface filters and aggregates data. For example, data can be filtered and arranged around an anchor point and/or other reference, for aggregation and formation into patient vectors.

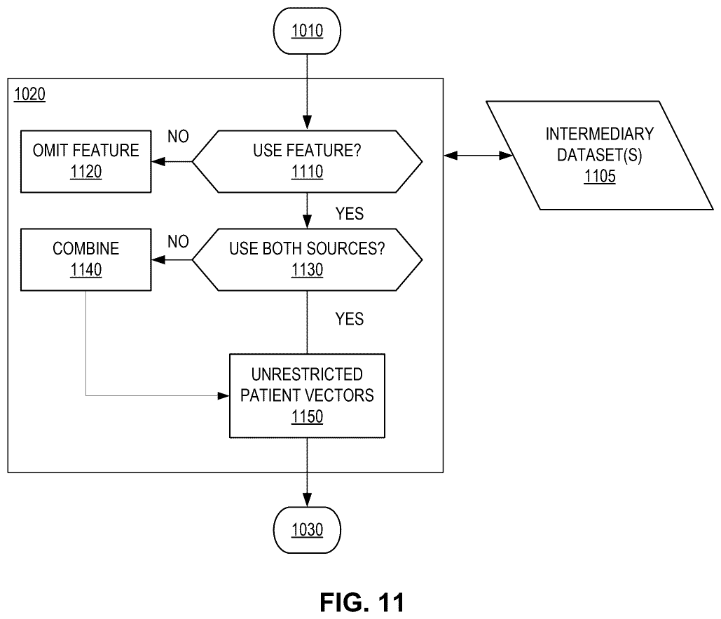

FIG. 11 provides an example implementation of generating patient vectors (e.g., block 1020 of the example process 1000). At block 1110, processed (e.g., filtered, cleaned, etc.) data is evaluated to determine whether a feature in the data is to be used? When the feature is not to be used, at block 1120, the feature is omitted. When the feature is to be used, at block 1130, available data (sources) are evaluated to determine whether multiple sources (e.g., structured and/or curated data sources, etc.) are to be used. When multiple sources are to be used, at block 1140, features from multiple data sources are combined and harmonized. At block 1150, unrestricted patient vectors are formed from downstream model-agnostic unaggregated tables retaining temporal structure. An intermediary dataset output 1105 can include one or more datasets separated by input domain and retaining temporal structure, for example.

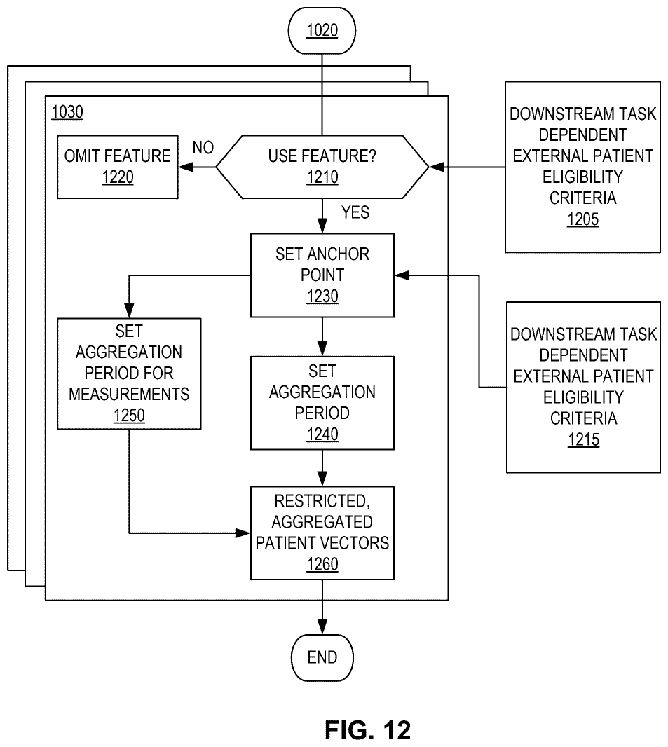

FIG. 12 provides an example implementation of a filtering and aggregating interface (e.g., block 1030 of the example process 1000). At 1210, a patient is evaluated to determine whether the patient is patient for further evaluation and processing. Such a determination can be based at least in part on downstream task-dependent external patient eligibility criteria 1205, for example. For example, does the patient satisfy certain health criteria, age criteria, condition criteria, likelihood of success, etc. When the patient is not eligible, at block 1220, the patient is omitted from further processing.

When the patient is eligible, at block 1230, an anchor point is set for further evaluation. Determination of the anchor point can be based on downstream, task-dependent, external patient eligibility criteria 1215, for example. The anchor point is a patient timeline event that aligns a plurality of patients for comparison. For example, events in a patient's life and/or medical history such as diagnosis, symptom, birth, hospitalization, initial treatment, etc., can drive anchor point selection by the framework to align time series data for relational processing and comparison. The anchor point can be a configurable parameter dependent on downstream tasks 1205, for example.

At block 1240, an aggregation period is set. For example, the framework can configure and/or otherwise set an aggregation period before a prediction point (e.g., the anchor point and/or other point in time and/or data for the patient). For example, an aggregation period can include an aggregation of patient data 200 days before a prediction time, etc.

At block 1250, an aggregation period for measurements is set. For examples, the interface framework allows establishment of a different aggregation period for laboratory measurements, other measurements, etc., since such measurements can be more dynamically changing.

At block 1260, one or more restricted, aggregated patient vectors are formed as an output of the interface framework. Patient vectors can be tailored to multiple downstream tasks, for example. In certain examples, for a given set of inputs, patient vectors can be dynamically generated as downstream tasks change.

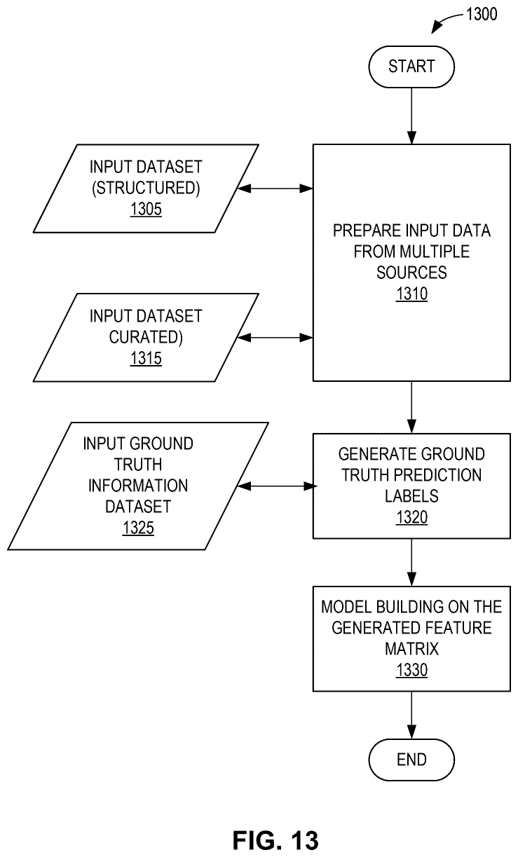

FIG. 13 illustrates an example process 1300 to build predictive models for efficacy related to ICI. Such models can assess multiple efficacy surrogate endpoints to predict a response to ICI therapies. Models can be created for separate cancer indications and/or combinations thereof.

At block 1310, input data from multiple sources is prepared for processing. For example, as described in more detail above, data can be cleaned and features extracted from sources such as a structured dataset 1305, a curated data set 1315, etc. For example, raw, automatically derived EHR data can be converted to a common format with curated data to form a set of features.

At block 1320, ground truth prediction labels are generated. Prediction labels can include time on ICI treatment (TOT), time to next treatment (TNET) (e.g., after discontinuation of ICI), overall survival (OS), etc. The listed ground truth endpoints can be derived from an input data 1325, for example, and can be generated on a continuous scale, such as expressed in days elapsed from an anchor point. For example, patient timelines can be aligned based on similarities in a course of ICI treatment. A default anchor point can be a first date of ICI treatment initiation, for example. Generated ground truth labels can be used as-is and/or with modified granularity (e.g., elapsed weeks, months, years, etc.) for training regression, survival analysis-based models, etc. Discretization of ground truth can be executed for binary and/or multi-class classification (e.g., responders versus non-responders, 5-year survival, etc.).

At block 1330, model building is conducted on a generated feature matrix in which the ground truth serves as a standalone module of the framework. In certain examples, efficacy endpoints can be modeled separately (e.g., individually, etc.). Modeling can be executed hypothesis-free and/or with different machine learning algorithms, for example. Alternatively or additionally, modeling can be done on a continuous scale with survival analysis methodology. Further, modeling can follow a multiclass classification methodology for all endpoints, for example. Example model building and selection is described further above.

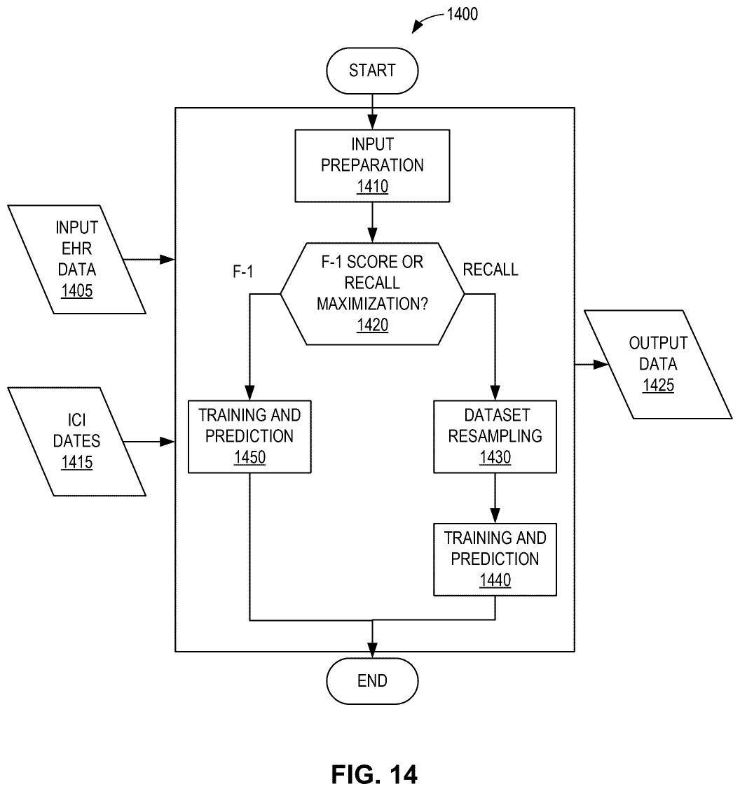

FIG. 14 illustrates an example method 1400 for model selection and generation. In the example of FIG. 14 model generation is driven at least in part by a purpose, goal, or target for the model. For example, the goal may be to generate a model having a high (e.g., maximized) F1 harmonic score (e.g., harmonic mean of recall and precision) such as a GB model, a model having high (e.g., maximized) recall (e.g., ratio of true positives found) such as a RF model, etc. In certain examples, majority class undersampling can be combined with time series data aggregation to obtain a well-balanced and static dataset to be fed to the models. In certain examples, models create probability estimates for labels, rather than only discrete labels themselves.

At block 1410, input is prepared, such as by extracting data from one or more EHR data tables 1405. Measurements such as liver biomarker concentration in blood plasma, other blood concentration values, etc., can be extracted. Time series measurement and/or other data can be put into a single complex data structure (e.g., aggregation), for example. Aggregated data can be processed using feature engineering to describe the time series data according to mean, standard deviation, minimum, maximum, etc. Lag and features and labels can be created, for example. In certain examples, a date of ICI treatment 1415 is generated to help align and anchor the time series data.

At block 1420, a target or goal for the model(s) is evaluated. As described above, goal of F1 maximization produces a different trained model (e.g., a gradient boosted model such as gradient-boosted decision tree, etc.) than a goal of recall maximization (e.g., a random forest model, etc.). Based on the determination, for recall maximization, at block 1430, dataset resampling occurs to balance unbalanced dataset from block 1410. For example, random majority class undersampling is performed on the dataset when the goal is to maximize the recall. When the goal is to maximize F-1 score, then resampling can be disregarded.

At block 1440 and/or 1450, a model is trained. For example, a RF model is trained for recall maximization at block 1440, and a GB model is trained for F1-score maximization at block 1450. The respective model is validated such as with leave-one-out cross-validation, for example, in which each sample is predicted individually with the rest serving as a training dataset.

While example implementations are illustrated in this application and associated appendix, one or more of the elements, processes, and/or devices illustrated may be combined, divided, re-arranged, omitted, eliminated, and/or implemented in any other way. Further, the example elements may be implemented by hardware alone or by hardware in combination with software and/or firmware. Thus, for example, any of the example elements could be implemented by processor circuitry, analog circuit(s), digital circuit(s), logic circuit(s), programmable processor(s), programmable microcontroller(s), graphics processing unit(s) (GPU(s)), digital signal processor(s) (DSP(s)), application specific integrated circuit(s) (ASIC(s)), programmable logic device(s) (PLD(s)), and/or field programmable logic device(s) (FPLD(s)) such as Field Programmable Gate Arrays (FPGAs). Further still, the example elements may include one or more elements, processes, and/or devices in addition to, or instead of, those illustrated, and/or may include more than one of any or all of the illustrated elements, processes and devices.

In certain examples, hardware logic circuitry, machine readable instructions, hardware implemented state machines, and/or any combination thereof can implement the system(s) and/or execute the methods disclosed herein. The machine readable instructions may be one or more executable programs or portion(s) of an executable program for execution by processor circuitry. The program may be embodied in software stored on one or more non-transitory computer readable storage media such as a compact disk (CD), a floppy disk, a hard disk drive (HDD), a solid-state drive (SSD), a digital versatile disk (DVD), a Blu-ray disk, a volatile memory (e.g., Random Access Memory (RAM) of any type, etc.), or a non-volatile memory (e.g., electrically erasable programmable read-only memory (EEPROM), FLASH memory, an HDD, an SSD, etc.) associated with processor circuitry located in one or more hardware devices, but the entire program and/or parts thereof could alternatively be executed by one or more hardware devices other than the processor circuitry and/or embodied in firmware or dedicated hardware. The machine readable instructions may be distributed across multiple hardware devices and/or executed by two or more hardware devices (e.g., a server and a client hardware device). For example, the client hardware device may be implemented by an endpoint client hardware device (e.g., a hardware device associated with a user) or an intermediate client hardware device (e.g., a radio access network (RAN)) gateway that may facilitate communication between a server and an endpoint client hardware device). Similarly, the non-transitory computer readable storage media may include one or more mediums located in one or more hardware devices. Further, an order of execution may be changed, and/or some of the blocks described may be changed, eliminated, or combined. Additionally or alternatively, any or all code blocks may be implemented by one or more hardware circuits (e.g., processor circuitry, discrete and/or integrated analog and/or digital circuitry, an FPGA, an ASIC, a comparator, an operational-amplifier (op-amp), a logic circuit, etc.) structured to perform the corresponding operation without executing software or firmware. The processor circuitry may be distributed in different network locations and/or local to one or more hardware devices (e.g., a single-core processor (e.g., a single core central processor unit (CPU)), a multi-core processor (e.g., a multi-core CPU, an XPU, etc.) in a single machine, multiple processors distributed across multiple servers of a server rack, multiple processors distributed across one or more server racks, a CPU and/or a FPGA located in the same package (e.g., the same integrated circuit (IC) package or in two or more separate housings, etc.).

The machine readable instructions described herein may be stored in one or more of a compressed format, an encrypted format, a fragmented format, a compiled format, an executable format, a packaged format, etc. Machine readable instructions as described herein may be stored as data or a data structure (e.g., as portions of instructions, code, representations of code, etc.) that may be utilized to create, manufacture, and/or produce machine executable instructions. For example, the machine readable instructions may be fragmented and stored on one or more storage devices and/or computing devices (e.g., servers) located at the same or different locations of a network or collection of networks (e.g., in the cloud, in edge devices, etc.). The machine readable instructions may require one or more of installation, modification, adaptation, updating, combining, supplementing, configuring, decryption, decompression, unpacking, distribution, reassignment, compilation, etc., in order to make them directly readable, interpretable, and/or executable by a computing device and/or other machine. For example, the machine readable instructions may be stored in multiple parts, which are individually compressed, encrypted, and/or stored on separate computing devices, wherein the parts when decrypted, decompressed, and/or combined form a set of machine executable instructions that implement one or more operations that may together form a program such as that described herein.

In another example, the machine readable instructions may be stored in a state in which they may be read by processor circuitry, but require addition of a library (e.g., a dynamic link library (DLL)), a software development kit (SDK), an application programming interface (API), etc., in order to execute the machine readable instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, machine readable media, as used herein, may include machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

As mentioned above, the example operations disclosed herein may be implemented using executable instructions (e.g., computer and/or machine readable instructions) stored on one or more non-transitory computer and/or machine readable media such as optical storage devices, magnetic storage devices, an HDD, a flash memory, a read-only memory (ROM), a CD, a DVD, a cache, a RAM of any type, a register, and/or any other storage device or storage disk in which information is stored for any duration (e.g., for extended time periods, permanently, for brief instances, for temporarily buffering, and/or for caching of the information). As used herein, the terms non-transitory computer readable medium, non-transitory computer readable storage medium, non-transitory machine readable medium, and non-transitory machine readable storage medium are expressly defined to include any type of computer readable storage device and/or storage disk and to exclude propagating signals and to exclude transmission media. As used herein, the terms “computer readable storage device” and “machine readable storage device” are defined to include any physical (mechanical and/or electrical) structure to store information, but to exclude propagating signals and to exclude transmission media. Examples of computer readable storage devices and machine readable storage devices include random access memory of any type, read only memory of any type, solid state memory, flash memory, optical discs, magnetic disks, disk drives, and/or redundant array of independent disks (RAID) systems. As used herein, the term “device” refers to physical structure such as mechanical and/or electrical equipment, hardware, and/or circuitry that may or may not be configured by computer readable instructions, machine readable instructions, etc., and/or manufactured to execute computer readable instructions, machine readable instructions, etc.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements or method actions may be implemented by, e.g., the same entity or object. Additionally, although individual features may be included in different examples or claims, these may possibly be combined, and the inclusion in different examples or claims does not imply that a combination of features is not feasible and/or advantageous.

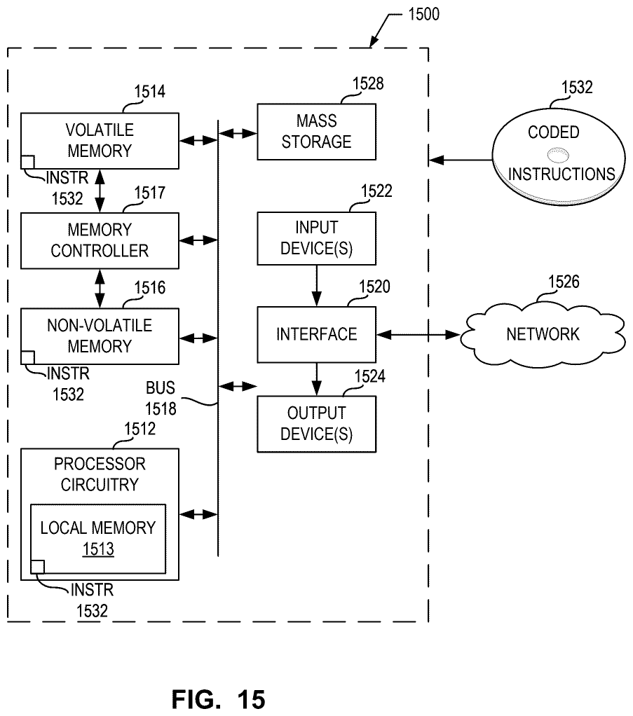

FIG. 15 is a block diagram of an example processor platform 1500 structured to execute and/or instantiate the machine readable instructions and/or the operations disclosed and described herein. The processor platform 1500 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset (e.g., an augmented reality (AR) headset, a virtual reality (VR) headset, etc.) or other wearable device, or any other type of computing device.

The processor platform 1500 of the illustrated example includes processor circuitry 1512. The processor circuitry 1512 of the illustrated example is hardware. For example, the processor circuitry 1512 can be implemented by one or more integrated circuits, logic circuits, FPGAs, microprocessors, CPUs, GPUs, DSPs, and/or microcontrollers from any desired family or manufacturer. The processor circuitry 1512 may be implemented by one or more semiconductor based (e.g., silicon based) devices.

The processor circuitry 1512 of the illustrated example includes a local memory 1513 (e.g., a cache, registers, etc.). The processor circuitry 1512 of the illustrated example is in communication with a main memory including a volatile memory 1514 and a non-volatile memory 1516 by a bus 1518. The volatile memory 1514 may be implemented by Synchronous Dynamic Random Access Memory (SDRAM), Dynamic Random Access Memory (DRAM), RAMBUS® Dynamic Random Access Memory (RDRAM®), and/or any other type of RAM device. The non-volatile memory 1516 may be implemented by flash memory and/or any other desired type of memory device. Access to the main memory 1514, 1516 of the illustrated example is controlled by a memory controller 1517.

The processor platform 1500 of the illustrated example also includes interface circuitry 1520. The interface circuitry 1520 may be implemented by hardware in accordance with any type of interface standard, such as an Ethernet interface, a universal serial bus (USB) interface, a Bluetooth® interface, a near field communication (NFC) interface, a Peripheral Component Interconnect (PCI) interface, and/or a Peripheral Component Interconnect Express (PCIe) interface.

In the illustrated example, one or more input devices 1522 are connected to the interface circuitry 1520. The input device(s) 1522 permit(s) a user to enter data and/or commands into the processor circuitry 1512. The input device(s) 1522 can be implemented by, for example, an audio sensor, a microphone, a camera (still or video), a keyboard, a button, a mouse, a touchscreen, a track-pad, a trackball, an isopoint device, and/or a voice recognition system.

One or more output devices 1524 are also connected to the interface circuitry 1520 of the illustrated example. The output device(s) 1524 can be implemented, for example, by display devices (e.g., a light emitting diode (LED), an organic light emitting diode (OLED), a liquid crystal display (LCD), a cathode ray tube (CRT) display, an in-place switching (IPS) display, a touchscreen, etc.), a tactile output device, a printer, and/or speaker. The interface circuitry 1520 of the illustrated example, thus, typically includes a graphics driver card, a graphics driver chip, and/or graphics processor circuitry such as a GPU.

The interface circuitry 1520 of the illustrated example also includes a communication device such as a transmitter, a receiver, a transceiver, a modem, a residential gateway, a wireless access point, and/or a network interface to facilitate exchange of data with external machines (e.g., computing devices of any kind) by a network 1526. The communication can be by, for example, an Ethernet connection, a digital subscriber line (DSL) connection, a telephone line connection, a coaxial cable system, a satellite system, a line-of-site wireless system, a cellular telephone system, an optical connection, etc.

The processor platform 1500 of the illustrated example also includes one or more mass storage devices 1528 to store software and/or data. Examples of such mass storage devices 1528 include magnetic storage devices, optical storage devices, floppy disk drives, HDDs, CDs, Blu-ray disk drives, redundant array of independent disks (RAID) systems, solid state storage devices such as flash memory devices and/or SSDs, and DVD drives.

The machine readable instructions 1532 may be stored in the mass storage device 1528, in the volatile memory 1514, in the non-volatile memory 1516, and/or on a removable non-transitory computer readable storage medium such as a CD or DVD.

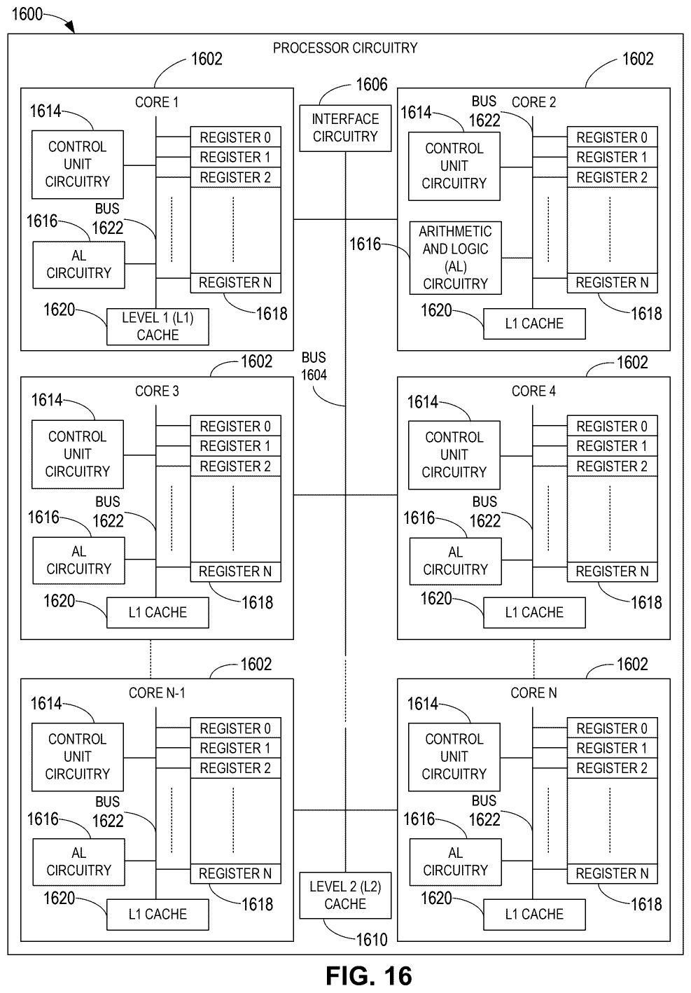

FIG. 16 is a block diagram of an example implementation of the processor circuitry 1512 of FIG. 15. In this example, the processor circuitry 1512 of FIG. 15 is implemented by a microprocessor 1600. For example, the microprocessor 1600 may be a general purpose microprocessor (e.g., general purpose microprocessor circuitry). The microprocessor 1600 executes some or all of the machine readable instructions to effectively instantiate the circuitry described herein as logic circuits to perform the operations corresponding to those machine readable instructions. In some such examples, the circuitry is instantiated by the hardware circuits of the microprocessor 1600 in combination with the instructions. For example, the microprocessor 1600 may be implemented by multi-core hardware circuitry such as a CPU, a DSP, a GPU, an XPU, etc. Although it may include any number of example cores 1602 (e.g., 1 core), the microprocessor 1600 of this example is a multi-core semiconductor device including N cores. The cores 1602 of the microprocessor 1600 may operate independently or may cooperate to execute machine readable instructions. For example, machine code corresponding to a firmware program, an embedded software program, or a software program may be executed by one of the cores 1602 or may be executed by multiple ones of the cores 1602 at the same or different times. In some examples, the machine code corresponding to the firmware program, the embedded software program, or the software program is split into threads and executed in parallel by two or more of the cores 1602. The software program may correspond to a portion or all of the machine readable instructions and/or operations disclosed herein.

The cores 1602 may communicate by a first example bus 1604. In some examples, the first bus 1604 may be implemented by a communication bus to effectuate communication associated with one(s) of the cores 1602. For example, the first bus 1604 may be implemented by at least one of an Inter-Integrated Circuit (I2C) bus, a Serial Peripheral Interface (SPI) bus, a PCI bus, or a PCIe bus. Additionally or alternatively, the first bus 1604 may be implemented by any other type of computing or electrical bus. The cores 1602 may obtain data, instructions, and/or signals from one or more external devices by example interface circuitry 1606. The cores 1602 may output data, instructions, and/or signals to the one or more external devices by the interface circuitry 1606. Although the cores 1602 of this example include example local memory 1620 (e.g., Level 1 (L1) cache that may be split into an L1 data cache and an L1 instruction cache), the microprocessor 1600 also includes example shared memory 1610 that may be shared by the cores (e.g., Level 2 (L2 cache)) for high-speed access to data and/or instructions. Data and/or instructions may be transferred (e.g., shared) by writing to and/or reading from the shared memory 1610. The local memory 1620 of each of the cores 1602 and the shared memory 1610 may be part of a hierarchy of storage devices including multiple levels of cache memory and the main memory (e.g., the main memory 1514, 1516 of FIG. 15). Typically, higher levels of memory in the hierarchy exhibit lower access time and have smaller storage capacity than lower levels of memory. Changes in the various levels of the cache hierarchy are managed (e.g., coordinated) by a cache coherency policy.

Each core 1602 may be referred to as a CPU, DSP, GPU, etc., or any other type of hardware circuitry. Each core 1602 includes control unit circuitry 1614, arithmetic and logic (AL) circuitry (sometimes referred to as an ALU) 1616, a plurality of registers 1618, the local memory 1620, and a second example bus 1622. Other structures may be present. For example, each core 1602 may include vector unit circuitry, single instruction multiple data (SIMD) unit circuitry, load/store unit (LSU) circuitry, branch/jump unit circuitry, floating-point unit (FPU) circuitry, etc. The control unit circuitry 1614 includes semiconductor-based circuits structured to control (e.g., coordinate) data movement within the corresponding core 1602. The AL circuitry 1616 includes semiconductor-based circuits structured to perform one or more mathematic and/or logic operations on the data within the corresponding core 1602. The AL circuitry 1616 of some examples performs integer based operations. In other examples, the AL circuitry 1616 also performs floating point operations. In yet other examples, the AL circuitry 1616 may include first AL circuitry that performs integer based operations and second AL circuitry that performs floating point operations. In some examples, the AL circuitry 1616 may be referred to as an Arithmetic Logic Unit (ALU). The registers 1618 are semiconductor-based structures to store data and/or instructions such as results of one or more of the operations performed by the AL circuitry 1616 of the corresponding core 1602. For example, the registers 1618 may include vector register(s), SIMD register(s), general purpose register(s), flag register(s), segment register(s), machine specific register(s), instruction pointer register(s), control register(s), debug register(s), memory management register(s), machine check register(s), etc. The registers 1618 may be arranged in a bank as shown in FIG. 16. Alternatively, the registers 1618 may be organized in any other arrangement, format, or structure including distributed throughout the core 1602 to shorten access time. The second bus 1622 may be implemented by at least one of an I2C bus, a SPI bus, a PCI bus, or a PCIe bus.

Each core 1602 and/or, more generally, the microprocessor 1600 may include additional and/or alternate structures to those shown and described above. For example, one or more clock circuits, one or more power supplies, one or more power gates, one or more cache home agents (CHAs), one or more converged/common mesh stops (CMSs), one or more shifters (e.g., barrel shifter(s)) and/or other circuitry may be present. The microprocessor 1600 is a semiconductor device fabricated to include many transistors interconnected to implement the structures described above in one or more integrated circuits (ICs) contained in one or more packages. The processor circuitry may include and/or cooperate with one or more accelerators. In some examples, accelerators are implemented by logic circuitry to perform certain tasks more quickly and/or efficiently than can be done by a general purpose processor. Examples of accelerators include ASICs and FPGAs such as those discussed herein. A GPU or other programmable device can also be an accelerator. Accelerators may be on-board the processor circuitry, in the same chip package as the processor circuitry and/or in one or more separate packages from the processor circuitry.

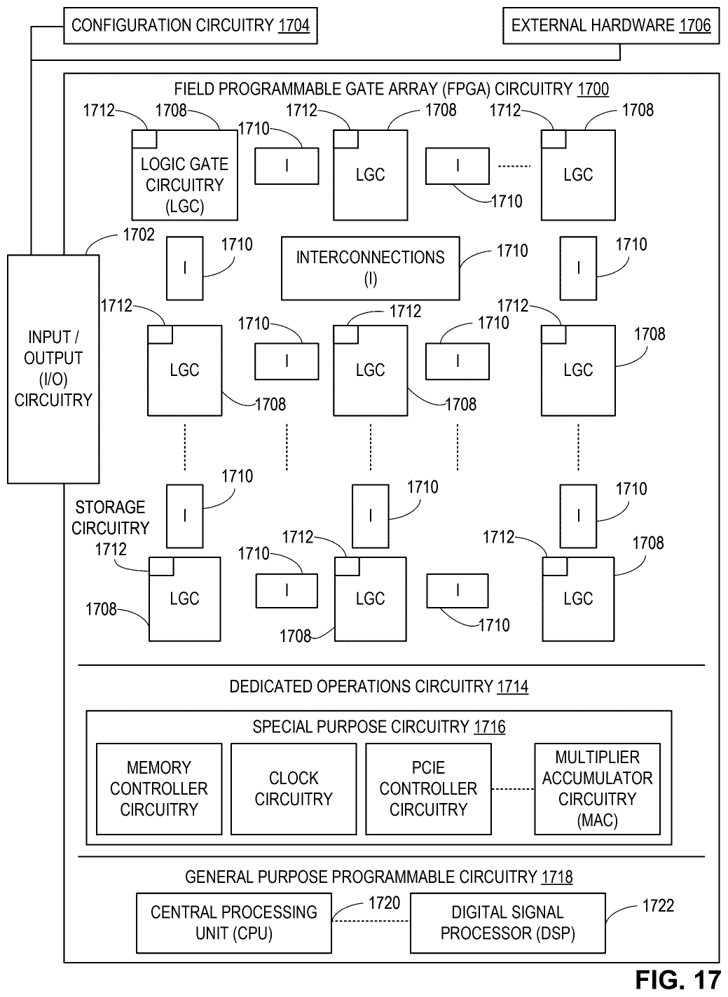

FIG. 17 is a block diagram of another example implementation of the processor circuitry 1512 of FIG. 15. In this example, the processor circuitry 1512 is implemented by FPGA circuitry 1700. For example, the FPGA circuitry 1700 may be implemented by an FPGA. The FPGA circuitry 1700 can be used, for example, to perform operations that could otherwise be performed by the example microprocessor 1600 of FIG. 16 executing corresponding machine readable instructions. However, once configured, the FPGA circuitry 1700 instantiates the machine readable instructions in hardware and, thus, can often execute the operations faster than they could be performed by a general purpose microprocessor executing the corresponding software.

More specifically, in contrast to the microprocessor 1600 of FIG. 16 described above (which is a general purpose device that may be programmed to execute some or all of the machine readable instructions disclosed here but whose interconnections and logic circuitry are fixed once fabricated), the FPGA circuitry 1700 of the example of FIG. 17 includes interconnections and logic circuitry that may be configured and/or interconnected in different ways after fabrication to instantiate, for example, some or all of the machine readable instructions disclosed herein. In particular, the FPGA circuitry 1700 may be thought of as an array of logic gates, interconnections, and switches. The switches can be programmed to change how the logic gates are interconnected by the interconnections, effectively forming one or more dedicated logic circuits (unless and until the FPGA circuitry 1700 is reprogrammed). The configured logic circuits enable the logic gates to cooperate in different ways to perform different operations on data received by input circuitry. Those operations may correspond to some or all of the software disclosed herein As such, the FPGA circuitry 1700 may be structured to effectively instantiate some or all of the machine readable instructions as dedicated logic circuits to perform the operations corresponding to those software instructions in a dedicated manner analogous to an ASIC. Therefore, the FPGA circuitry 1700 may perform the operations corresponding to the some or all of the machine readable instructions faster than the general purpose microprocessor can execute the same.

In the example of FIG. 17, the FPGA circuitry 1700 is structured to be programmed (and/or reprogrammed one or more times) by an end user by a hardware description language (HDL) such as Verilog. The FPGA circuitry 1700 of FIG. 17, includes example input/output (I/O) circuitry 1702 to obtain and/or output data to/from example configuration circuitry 1704 and/or external hardware 1706. For example, the configuration circuitry 1704 may be implemented by interface circuitry that may obtain machine readable instructions to configure the FPGA circuitry 1700, or portion(s) thereof. In some such examples, the configuration circuitry 1704 may obtain the machine readable instructions from a user, a machine (e.g., hardware circuitry (e.g., programmed or dedicated circuitry) that may implement an Artificial Intelligence/Machine Learning (AI/ML) model to generate the instructions), etc. In some examples, the external hardware 1706 may be implemented by external hardware circuitry. For example, the external hardware 1706 may be implemented by the microprocessor 1600 of FIG. 16. The FPGA circuitry 1700 also includes an array of example logic gate circuitry 1708, a plurality of example configurable interconnections 1710, and example storage circuitry 1712. The logic gate circuitry 1708 and the configurable interconnections 1710 are configurable to instantiate one or more operations that may correspond to at least some of the machine readable instructions and/or other desired operations. The logic gate circuitry 1708 shown in FIG. 17 is fabricated in groups or blocks. Each block includes semiconductor-based electrical structures that may be configured into logic circuits. In some examples, the electrical structures include logic gates (e.g., And gates, Or gates, Nor gates, etc.) that provide basic building blocks for logic circuits. Electrically controllable switches (e.g., transistors) are present within each of the logic gate circuitry 1708 to enable configuration of the electrical structures and/or the logic gates to form circuits to perform desired operations. The logic gate circuitry 1708 may include other electrical structures such as look-up tables (LUTs), registers (e.g., flip-flops or latches), multiplexers, etc.

The configurable interconnections 1710 of the illustrated example are conductive pathways, traces, vias, or the like that may include electrically controllable switches (e.g., transistors) whose state can be changed by programming (e.g., using an HDL instruction language) to activate or deactivate one or more connections between one or more of the logic gate circuitry 1708 to program desired logic circuits.

The storage circuitry 1712 of the illustrated example is structured to store result(s) of the one or more of the operations performed by corresponding logic gates. The storage circuitry 1712 may be implemented by registers or the like. In the illustrated example, the storage circuitry 1712 is distributed amongst the logic gate circuitry 1708 to facilitate access and increase execution speed.

The example FPGA circuitry 1700 of FIG. 17 also includes example Dedicated Operations Circuitry 1714. In this example, the Dedicated Operations Circuitry 1714 includes special purpose circuitry 1716 that may be invoked to implement commonly used functions to avoid the need to program those functions in the field. Examples of such special purpose circuitry 1716 include memory (e.g., DRAM) controller circuitry, PCIe controller circuitry, clock circuitry, transceiver circuitry, memory, and multiplier-accumulator circuitry. Other types of special purpose circuitry may be present. In some examples, the FPGA circuitry 1700 may also include example general purpose programmable circuitry 1718 such as an example CPU 1720 and/or an example DSP 1722. Other general purpose programmable circuitry 1718 may additionally or alternatively be present such as a GPU, an XPU, etc., that can be programmed to perform other operations.

Although FIGS. 16 and 17 illustrate two example implementations of the processor circuitry 1512 of FIG. 15, many other approaches are contemplated. For example, as mentioned above, modern FPGA circuitry may include an on-board CPU, such as one or more of the example CPU 1720 of FIG. 17. Therefore, the processor circuitry 1512 of FIG. 15 may additionally be implemented by combining the example microprocessor 1600 of FIG. 16 and the example FPGA circuitry 1700 of FIG. 17. In some such hybrid examples, a first portion of the machine readable instructions may be executed by one or more of the cores 1602 of FIG. 16, a second portion of the machine readable instructions may be executed by the FPGA circuitry 1700 of FIG. 17, and/or a third portion of the machine readable instructions may be executed by an ASIC. It should be understood that some or all of the circuitry may, thus, be instantiated at the same or different times. Some or all of the circuitry may be instantiated, for example, in one or more threads executing concurrently and/or in series. Moreover, in some examples, some or all of the circuitry may be implemented within one or more virtual machines and/or containers executing on the microprocessor.

In some examples, the processor circuitry 1512 of FIG. 15 may be in one or more packages. For example, the microprocessor 1600 of FIG. 16 and/or the FPGA circuitry 1700 of FIG. 17 may be in one or more packages. In some examples, an XPU may be implemented by the processor circuitry 1512 of FIG. 15, which may be in one or more packages. For example, the XPU may include a CPU in one package, a DSP in another package, a GPU in yet another package, and an FPGA in still yet another package.

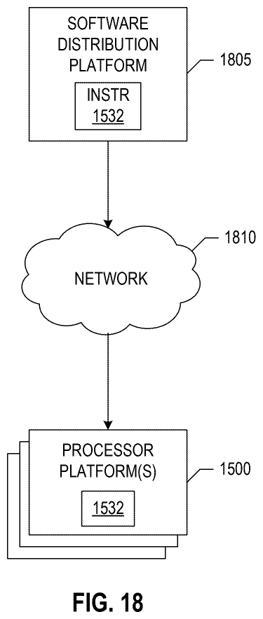

A block diagram illustrating an example software distribution platform 1805 to distribute software such as the example machine readable instructions 1532 of FIG. 15 to hardware devices owned and/or operated by third parties is illustrated in FIG. 18. The example software distribution platform 1805 may be implemented by any computer server, data facility, cloud service, etc., capable of storing and transmitting software to other computing devices. The third parties may be customers of the entity owning and/or operating the software distribution platform 1805. For example, the entity that owns and/or operates the software distribution platform 1805 may be a developer, a seller, and/or a licensor of software such as the example machine readable instructions 1532 of FIG. 15. The third parties may be consumers, users, retailers, OEMs, etc., who purchase and/or license the software for use and/or re-sale and/or sub-licensing. In the illustrated example, the software distribution platform 1805 includes one or more servers and one or more storage devices. The storage devices store the machine readable instructions 1532, which may correspond to the example machine readable instructions 1532 of FIG. 15, as described above. The one or more servers of the example software distribution platform 1805 are in communication with an example network 1810, which may correspond to any one or more of the Internet and/or any of the example networks described above. In some examples, the one or more servers are responsive to requests to transmit the software to a requesting party as part of a commercial transaction. Payment for the delivery, sale, and/or license of the software may be handled by the one or more servers of the software distribution platform and/or by a third party payment entity. The servers enable purchasers and/or licensors to download the machine readable instructions 1532 from the software distribution platform 1805. For example, the software, which may correspond to the example machine readable instructions 1532 of FIG. 15, may be downloaded to the example processor platform 1500, which is to execute the machine readable instructions 1532 to implement the systems and methods described herein. In some examples, one or more servers of the software distribution platform 1805 periodically offer, transmit, and/or force updates to the software (e.g., the example machine readable instructions 1532) to ensure improvements, patches, updates, etc., are distributed and applied to the software at the end user devices.

In some examples, rather than downloading machine readable instructions 1832 to a local processor platform 1800, a deployed model and/or patient data can be uploaded to execute remotely via the cloud-based platform 1805. In some examples, the example platform 1805 can host one or more models, accessible by the network 1810, and a processor platform 1500 can provide input to the model and receive a result, prediction, and/or other output.

From the foregoing, it will be appreciated that example systems, methods, apparatus, and articles of manufacture have been disclosed that enable model generation and deployment to drive processes for therapeutic prediction and treatment execution. Disclosed systems, methods, apparatus, and articles of manufacture improve the efficiency of using a computing device by enabling model generation and deployment to drive processes for therapeutic prediction and treatment execution. Disclosed systems, methods, apparatus, and articles of manufacture are accordingly directed to one or more improvement(s) in the operation of a machine such as a computer or other electronic and/or mechanical device.

Certain examples provide systems and methods to train and evaluate an AI model to predict an adverse risk event from fixed length patient history data. An example system and associated method include a submodule to extract patient blood test histories from electronic medical records, clean the histories and perform data quality checks. The example system and associated method include a label definition submodule instantiating an algorithm to assign a hepatitis adverse event grade to a set of blood test values (e.g., ALT, AST, TBILIRUBIN, ALKPHOS), and create a binary target label for the AI model. The example system and method include a feature engineering submodule to normalize blood test values to the upper limit of normal value (e.g., specific to patient, laboratory, and/or blood test, etc.). The example feature engineering submodule is to transform the normalized values to a discretized symbolic representation, such as a modified version of Symbolic Aggregate ApproXimation, etc. The example feature engineering submodule is to extract motifs as n-grams from the symbol series, and use the counts in recent patient history as features. The example system and method include a submodule to train and evaluate an AI model to dynamically predict immune-related hepatitis adverse event risk from fixed length blood test histories.

Certain examples provide systems and methods to build a classification model (e.g., a pneumonitis classification model, etc.) using a sequential procedure. An example system and method include preprocessing structured EHR and unstructured data tables. Patient timelines are aligned at the first ICI administration, for example. Lab measurements are aggregated over a time window (e.g., a 60-day time window, etc.) before the first ICI using statistics. Other features (e.g., conditions, smoking status, etc.) can use a different time window (e.g., a 1-year time window, etc.), for example.

The example system and method include finding patterns in the data to identify potential predictive features associated with development of ICI-related toxicities like pneumonitis. In case of a small and noisy dataset, many data points are utilized. The data is split, with a first partition (e.g., a 90% partition, an 80% partition, a 95% partition, etc.) to identify candidate features based on associations between the pneumonitis label and the features.

The example system and method include, in each iteration of the procedure, deciding between two model versions, one with the original feature set (M1) and one extended with a candidate feature (M2). Nested cross-validation is performed on the first (e.g., 90%, etc.) partition, and the inner loop results are used to compare M1 and M2. A binomial test is performed to assess whether M2 is significantly better than M1.

The example system and method include, when M2 is significantly better in step 3), assessing whether M2 has better performance on the held out second partition (e.g., a 10% partition, 5% partition, 20% partition, etc.). A permutation test is performed to estimate the probability of observing a better performance just by random chance. This step acts as a safety measure to avoid overfitting to the first (e.g., 90%, etc.) data partition. If M2 has sufficiently better performance based on step 4), M2 is chosen.

The example system and method include continuing to test new candidate features until a desired model size is reached. The final model's performance on the outer loop is assessed. This way, a performance estimator with smaller variance is obtained, and variability in test predictions and model instability can be assessed. If the final model has promising performance, the model is evaluated on an external test set that is sufficiently large.

Certain examples provide systems and methods forming an automated framework to prepare multiple source electronic health record data for use in machine learning model training. The example method and associated system include preparing input data from multiple sources: This part of the framework is mainly concerned about cleaning and extracting features from multiple data sources. The step takes in raw, automatically derived EHR data in a data model format (e.g., an OMOP Data Model format, etc.) and multiple expert curated data sources for additional features and labels. The step is open for extensions and includes but is not restricted to modules for preparation of smoking history, drug administration, medical conditions, radiotherapy history, laboratory measurements and anthropometric data.

The example method and associated system include generating combined time dependent unrestricted intermediary outputs. This step is condensing the input data in a uniform format, retaining time stamps of the individual data items per patient. This intermediary step provides a plug-in possibility to modules preparing time dependent input data (not implemented) for sequence modeling algorithms and provides a flexible input for the aggregation step of the framework.

The example method and associated system include aggregating features for time independent models. This step is a target agnostic, highly configurable plug-in module for creating time independent, aggregated inputs for machine learning models. The step is open for extensions, configurable parameters include but are not restricted to prediction time point, length of aggregation, data sources to involve, feature types to involve.

Certain examples provide systems and methods for predictive model building for efficacy surrogate endpoint related to immune checkpoint inhibitor treatment. An example method and associated system include preparing input data from multiple sources. This part of the framework involves cleaning and extracting features from multiple data sources. The step takes in raw, automatically derived EHR data in OMOP Data Model format, for example, and multiple expert curated data sources for additional features. The example method and associated system include generating ground truth prediction labels such as Time on ICI treatment (TOT), Time to next treatment (after ICI discontinuation) (TNET), Overall Survival (OS), etc. The listed ground truth endpoints are generated on a continuous scale, expressed in days elapsed from an anchor point (patient timelines are alignment based on similarities in the ICI treatment course). The default anchor point is the first date of ICI treatment initiation. Generated ground truth can be used as is, or with modified granularity (elapsed weeks, months, years etc.) for training regression or survival analysis-based models. Discretization of the ground truth can be carried out for binary, or multiclass classification (e.g., responders vs. non-responders, 5-year survival, etc.).

The example method and associated system include model building on the generated feature matrix and the ground truth as a standalone module of the framework. Endpoints can be modeled separately. The modelling can be carried out hypothesis free, and with different machine learning algorithms, for example. A model building and selection workflow can be used to generate a predictive model for immune checkpoint inhibitor-related pneumonitis and a sequential procedure for model building.

Certain examples provide systems and methods for hepatitis prediction. An example system and associated method include input preparation through extraction of the relevant section of blood features from the Electronic Health Record (EHR) data tables received. These are measurements of liver biomarker concentration in the blood plasma (such as ALT, AST, Alkaline Phosphatase and Bilirubin) and other concentration values in the blood. This step is followed by putting the time series data into a single complex data structure, which is an efficient option to then continue by aggregating this information into the final data table, which is now ready for preprocessing and transformation steps. The aggregation step of the feature engineering consists of describing the time-series data of the blood particles with their mean, standard deviation, minimum and maximum. Lag features can also be created by taking the last liver biomarker measurements available before the treatment. The labels are created using a definition obtained from medical experts, which, for example, classified someone as positive when the level of at least one liver biomarker exceeded 3-times the upper limit of normal within a predefined window, negative otherwise. The date of the ICI treatment used in this scheme can be output from a different workflow.

The example system and method include dataset resampling. For example, the resulting dataset from step 1 is unbalanced, therefore random majority class us. is performed on the dataset if the goal is to maximize the recall value. If the F1-score is the subject of maximization, then the resampling step is disregarded.

The example system and method include training and prediction. For example, on the final data resulted from step 2, a model is trained, which is RF for recall maximization, and GB for F1-score maximization. The validation is carried out with Leave-One-Out Cross-Validation, where each sample is predicted individually with the rest as the training dataset.

Further aspects of the present disclosure are provided by the subject matter of the following clauses:

Example 1 is an apparatus including: memory circuitry; instructions; and processor circuitry to execute the instructions to: process input data pulled from a record to form a set of candidate features; train at least a first model and a second model using the set of candidate features; test at least the first model and the second model to compare performance of the first model and the second model; select at least one of the first model or the second model based on the comparison; store the selected at least one of the first model or the second model; and deploy the selected at least one of the first model or the second model to predict a likelihood of at least one of: a) a toxicity occurring due to immunotherapy according to a treatment plan or b) efficacy of the treatment plan for a patient.

Example 2 includes the apparatus of any preceding clause, further including deploying the selected at least one of the first model or the second model in a tool with an interface to facilitate gathering of patient data and interaction with the selected at least one of the first model or the second model.

Example 3 includes the apparatus of any preceding clause, wherein the input data includes at least one of laboratory test results, diagnosis code, or billing codes.

Example 4 includes the apparatus of any preceding clause, wherein the toxicity includes at least one of pneumonitis, colitis, or hepatitis.

Example 5 includes the apparatus of any preceding clause, wherein the efficacy of the treatment plan for the patient is measured by at least one of patient survival or time on treatment.

Example 6 includes the apparatus of any preceding clause, wherein the processor circuitry is to extract and organize the input data in a time series.

Example 7 includes the apparatus of any preceding clause, wherein the processor circuitry is to align the input data with respect to an anchor point to organize the input data in the time series.

Example 8 includes the apparatus of any preceding clause, wherein the processor circuitry is to generate labels for the input data to form the set of candidate features.

Example 9 includes the apparatus of any preceding clause, wherein the processor circuitry is to feature engineer the set of candidate features by at least one of normalizing, transforming, or extracting from the set of candidate features.

Example 10 includes the apparatus of any preceding clause, wherein the processor circuitry is to select from the set of candidate features to form a patient feature set to at least one of train or test at least the first model and the second model based on the feature engineering.

Example 11 includes the apparatus of any preceding clause, wherein the processor circuitry is to generate a feature matrix to at least one of train or test at least the first model and the second model based on the feature engineering.

Example 12 includes the apparatus of any preceding clause, wherein the processor circuitry is to deploy the selected at least one of the first model or the second model as an executable tool with an interface.

Example 13 includes at least one computer-readable storage medium including instructions which, when executed by processor circuitry, cause the processor circuitry to at least: process input data pulled from a record to form a set of candidate features; train at least a first model and a second model using the set of candidate features; test at least the first model and the second model to compare performance of the first model and the second model; select at least one of the first model or the second model based on the comparison; store the selected at least one of the first model or the second model; and deploy the selected at least one of the first model or the second model to predict a likelihood of at least one of: a) a toxicity occurring due to immunotherapy according to a treatment plan or b) efficacy of the treatment plan for a patient.

Example 14 includes the at least one computer-readable storage medium of any preceding clause, wherein the instructions, when executed, cause the processor circuitry to deploy the selected at least one of the first model or the second model in a tool with an interface to facilitate gathering of patient data and interaction with the selected at least one of the first model or the second model.

Example 15 includes the at least one computer-readable storage medium of any preceding clause, wherein the instructions, when executed, cause the processor circuitry to extract and organize the input data in a time series with respect to an anchor point.

Example 16 includes the at least one computer-readable storage medium of any preceding clause, wherein the instructions, when executed, cause the processor circuitry to generate labels for the input data to form the set of candidate features.

Example 17 includes the at least one computer-readable storage medium of any preceding clause, wherein the instructions, when executed, cause the processor circuitry to feature engineer the set of candidate features by at least one of normalizing, transforming, or extracting from the set of candidate features.

Example 18 is a computer-implemented method including: processing input data pulled from a record to form a set of candidate features; training at least a first model and a second model using the set of candidate features; testing at least the first model and the second model to compare performance of the first model and the second model; selecting at least one of the first model or the second model based on the comparison; storing the selected at least one of the first model or the second model; and deploying the selected at least one of the first model or the second model to predict a likelihood of at least one of: a) a toxicity occurring due to immunotherapy according to a treatment plan or b) efficacy of the treatment plan for a patient.

Example 19 includes the method of any preceding clause, wherein the deploying includes deploying the selected at least one of the first model or the second model in a tool with an interface to facilitate gathering of patient data and interaction with the selected at least one of the first model or the second model.

Example 20 includes the method of any preceding clause, further including extracting and organizing the input data in a time series with respect to an anchor point.

Example 21 includes the method of any preceding clause, further including generating labels for the input data to form the set of candidate features.

Example 22 includes the method of any preceding clause, further including feature engineering the set of candidate features by at least one of normalizing, transforming, or extracting from the set of candidate features.

Example 23 is an apparatus including: memory circuitry; instructions; a plurality of models to predict at least one of a) a toxicity in response to immunotherapy or b) an efficacy of the immunotherapy, the plurality of models trained and validated using data from previous patients; and processor circuitry to execute the instructions to: accept an input, via an interface, of data associated with a first patient; generate, using at least one of the plurality of models, a prediction of at least one of: a) a toxicity occurring during immunotherapy according to a treatment plan for the first patient or b) an efficacy of the treatment plan for the first patient; and output a recommendation for the first patient with respect to the treatment plan.

Example 24 includes the apparatus of any preceding clause, wherein the toxicity includes at least one of pneumonitis, colitis, or hepatitis.

Example 25 includes the apparatus of any preceding clause, wherein efficacy is defined with respect to patient survival.

Example 26 includes the apparatus of any preceding clause, wherein efficacy is measured by either progression-free survival or by time on treatment.

Example 27 includes the apparatus of any preceding clause, wherein predictions of both toxicity and efficacy are generated for the first patient to enable assessment of a risk versus benefit ratio for the first patient with respect to the treatment plan.

Example 28 includes the apparatus of any preceding clause, wherein the processor circuitry is to extract and organize the data associated with the patient in a time series to be provided to the model.

Example 29 includes the apparatus of any preceding clause, wherein the processor circuitry is to align the data associated with the patient with respect to an anchor point to organize the patient data in the time series.

Example 30 includes the apparatus of any preceding clause, wherein the treatment plan includes a clinical trial involving the first patient.

Example 31 includes the apparatus of any preceding clause, wherein the treatment plan is part of clinical care for the first patient.

Example 32 includes the apparatus of any preceding clause, wherein the input is a first input of data at a first time and the prediction is a first prediction, and wherein the processor circuitry is to process a second input of data from the first patient at a second time to generate a second prediction with the model, the processor circuitry to compare the second prediction and the first prediction to adjust the recommendation output for the patient.

Example 33 includes the apparatus of any preceding clause, wherein the first prediction is used by a least one of the plurality of models to generate the second prediction.

Example 34 includes the apparatus of any preceding clause, wherein the processor circuitry is to compare the second prediction, the first prediction, and image data to adjust the recommendation that is output for the patient.

Example 35 includes the apparatus of any preceding clause, further including interface circuitry to connect to an electronic medical record to at least one of retrieve the data associated with the first patient or store the prediction.

Example 36 includes the apparatus of any preceding clause, wherein the processor circuitry is to obtain feedback regarding the recommendation to adjust the model.

Example 37 includes at least one computer-readable storage medium including instructions which, when executed by processor circuitry, cause the processor circuitry to at least: accept an input, via an interface, of data associated with a first patient; generate, using at least one of a plurality of models, a prediction of at least one of: a) a toxicity occurring during immunotherapy according to a treatment plan for the first patient or b) an efficacy of the treatment plan for the first patient, the plurality of models to predict at least one of a) a toxicity in response to immunotherapy or b) an efficacy of the immunotherapy, the plurality of models trained and validated using data from previous patients; and output a recommendation for the first patient with respect to the treatment plan.

Example 38 includes the at least one computer-readable storage medium of any preceding clause, wherein the instructions, when executed, cause the processor circuitry to extract and organize the patient data in a time series to be provided to the model.

Example 39 includes the at least one computer-readable storage medium of any preceding clause, wherein the instructions, when executed, cause the processor circuitry to align the patient data with respect to an anchor point to organize the patient data in the time series.

Example 40 includes the at least one computer-readable storage medium of any preceding clause, wherein the input is a first input of data at a first time and the prediction is a first prediction, and wherein the processor circuitry is to process a second input of data from the first patient at a second time to generate a second prediction with the model, the processor circuitry to compare the second prediction and the first prediction to adjust the recommendation output for the patient.

Example 41 is a method including: accepting an input, via an interface, of data associated with a first patient; generating, by executing an instruction using a processor and at least one of a plurality of models, a prediction of at least one of: a) a toxicity occurring during immunotherapy according to a treatment plan for the first patient or b) an efficacy of the treatment plan for the first patient, the plurality of models to predict at least one of a) a toxicity in response to immunotherapy or b) an efficacy of the immunotherapy, the plurality of models trained and validated using data from previous patients; and outputting, by executing an instruction using the processor, a recommendation for the first patient with respect to the treatment plan.

Example 42 includes the method of any preceding clause, further including extracting and organizing the patient data in a time series to be provided to the model.

Example 43 includes the method of any preceding clause, further including aligning the patient data with respect to an anchor point to organize the patient data in the time series.

Example 44 includes the method of any preceding clause, wherein the input is a first input of data at a first time and the prediction is a first prediction, and further including processing a second input of data from the first patient at a second time to generate a second prediction with the model; and comparing the second prediction and the first prediction to adjust the recommendation output for the patient.

Example 45 is an apparatus including means for processing input data pulled from a record to form a set of candidate features; means for training at least a first model and a second model using the set of candidate features; testing at least the first model and the second model to compare performance of the first model and the second model; means for selecting at least one of the first model or the second model based on the comparison; means for storing the selected at least one of the first model or the second model; and means for deploying the selected at least one of the first model or the second model to predict a likelihood of at least one of: a) a toxicity occurring due to immunotherapy according to a treatment plan or b) efficacy of the treatment plan for a patient.

As disclosed and described herein, processor circuitry provides a means for processing (e.g., including means for processing input data, means for training models, means for selecting, means for deploying, etc.) and memory circuitry provides a means for storing. As described above, various circuitry can be implemented by the processor circuitry and by the memory circuitry.

The following claims are hereby incorporated into this Detailed Description by this reference. Although certain example systems, methods, apparatus, and articles of manufacture have been disclosed herein, the scope of coverage of this patent is not limited thereto. On the contrary, this patent covers all systems, methods, apparatus, and articles of manufacture fairly falling within the scope of the claims of this patent.

What is claimed is:

1. An apparatus comprising:
memory circuitry;
instructions; and
processor circuitry to execute the instructions to:
process input data pulled from a record to form a set of candidate features;
train at least a first model and a second model using the set of candidate features;
test at least the first model and the second model to compare performance of the first model and the second model;
select at least one of the first model or the second model based on the comparison;
store the selected at least one of the first model or the second model; and
deploy the selected at least one of the first model or the second model to predict a likelihood of at least one of: a) a toxicity occurring due to immunotherapy according to a treatment plan or b) efficacy of the treatment plan for a patient.

2. The apparatus of claim 1, further including deploying the selected at least one of the first model or the second model in a tool with an interface to facilitate gathering of patient data and interaction with the selected at least one of the first model or the second model.

3. The apparatus of claim 1, wherein the input data includes at least one of laboratory test results, diagnosis code, or billing codes.

4. The apparatus of claim 1, wherein the toxicity includes at least one of pneumonitis, colitis, or hepatitis.

5. The apparatus of claim 1, wherein the efficacy of the treatment plan for the patient is measured by at least one of patient survival or time on treatment.

6. The apparatus of claim 1, wherein the processor circuitry is to extract and organize the input data in a time series.

7. The apparatus of claim 6, wherein the processor circuitry is to align the input data with respect to an anchor point to organize the input data in the time series.

8. The apparatus of claim 1, wherein the processor circuitry is to generate labels for the input data to form the set of candidate features.

9. The apparatus of claim 1, wherein the processor circuitry is to feature engineer the set of candidate features by at least one of normalizing, transforming, or extracting from the set of candidate features.

10. The apparatus of claim 9, wherein the processor circuitry is to select from the set of candidate features to form a patient feature set to at least one of train or test at least the first model and the second model based on the feature engineering.

11. The apparatus of claim 9, wherein the processor circuitry is to generate a feature matrix to at least one of train or test at least the first model and the second model based on the feature engineering.

12. The apparatus of claim 1, wherein the processor circuitry is to deploy the selected at least one of the first model or the second model as an executable tool with an interface.

13. At least one computer-readable storage medium comprising instructions which, when executed by processor circuitry, cause the processor circuitry to at least:
process input data pulled from a record to form a set of candidate features;
train at least a first model and a second model using the set of candidate features;
test at least the first model and the second model to compare performance of the first model and the second model;
select at least one of the first model or the second model based on the comparison;
store the selected at least one of the first model or the second model; and
deploy the selected at least one of the first model or the second model to predict a likelihood of at least one of: a) a toxicity occurring due to immunotherapy according to a treatment plan or b) efficacy of the treatment plan for a patient.

14. The at least one computer-readable storage medium of claim 13, wherein the instructions, when executed, cause the processor circuitry to deploy the selected at least one of the first model or the second model in a tool with an interface to facilitate gathering of patient data and interaction with the selected at least one of the first model or the second model.

15. The at least one computer-readable storage medium of claim 13, wherein the instructions, when executed, cause the processor circuitry to extract and organize the input data in a time series with respect to an anchor point.

16. The at least one computer-readable storage medium of claim 13, wherein the instructions, when executed, cause the processor circuitry to generate labels for the input data to form the set of candidate features.

17. The at least one computer-readable storage medium of claim 13, wherein the instructions, when executed, cause the processor circuitry to feature engineer the set of candidate features by at least one of normalizing, transforming, or extracting from the set of candidate features.

18. A computer-implemented method comprising:
processing input data pulled from a record to form a set of candidate features;
training at least a first model and a second model using the set of candidate features;
testing at least the first model and the second model to compare performance of the first model and the second model;
selecting at least one of the first model or the second model based on the comparison;
storing the selected at least one of the first model or the second model; and
deploying the selected at least one of the first model or the second model to predict a likelihood of at least one of: a) a toxicity occurring due to immunotherapy according to a treatment plan or b) efficacy of the treatment plan for a patient.

19. The method of claim 18, wherein the deploying includes deploying the selected at least one of the first model or the second model in a tool with an interface to facilitate gathering of patient data and interaction with the selected at least one of the first model or the second model.

20. The method of claim 18, further including extracting and organizing the input data in a time series with respect to an anchor point.

21. The method of claim 18, further including generating labels for the input data to form the set of candidate features.

22. The method of claim 18, further including feature engineering the set of candidate features by at least one of normalizing, transforming, or extracting from the set of candidate features.
